(19) United States
(12) Patent Application Publication
(10) Pub. No.: US 2026/0152554 A1
(43) Pub. Date: Jun. 4, 2026

(54) HUMANIZED ANTIBODIES AGAINST CD79B

(71) Applicant: ATB THERAPEUTICS, Aye (BE)

(72) Inventors: Max HOURY, Saint-Denis (BE); Bertrand MAGY, Marche-les-Dames (BE); Sergej KIPRIJANOV, Halberstadt (DE)

(21) Appl. No.: 19/101,198

(22) PCT Filed: Aug. 8, 2023
(86) PCT No.: PCT/EP2023/071934

§ 371 Date: Feb. 4, 2025

(30) Foreign Application Priority Data
Aug. 8, 2022 (EP) 22189322.5

(51) Int. Cl. C07K 16/28; A61P 35/00

(52) U.S. Cl. CPC C07K 16/2803; A61P 35/00; C07K 2317/24; C07K 2317/33; C07K 2319/55

(57) ABSTRACT

The present invention relates to humanized antibodies or target-binding fragments or derivatives thereof retaining target binding capacities, which bind to human CD79b (FIG. 1)

### SEQUENCE LISTING

The content of the electronically submitted sequence listing (Name: 5648_0040001_SequenceListing_ST26.xml; Size: 59,627 bytes; Date of Creation: Jan. 9, 2026) filed with the application is incorporated herein by reference in its entirety.

### FIELD OF THE INVENTION

The present application relates to humanized antibodies against CD79b

### BACKGROUND

Despite fundamental progress in the recent past, many cancer types are still difficult to impossible to treat successfully. This applies, in particular to B-cell related malignancies. Here, targeted therapies exist against, inter alia, CD20 and CD19, and CD22.

It has been shown that CD79b (immunoglobulin-associated beta), which is likewise an antigen on B-cells, may offer advantages, in its use as a target, over inter alia CD20

CD79b, along with CD79a and a surface immunoglobulin, make up the -cell receptor BCR), which is expressed on over 90% of B-cell NHL malignancies. CD79b can be internalized upon antibody binding. Due to this functionality, CD79b has the potential to selectively deliver molecules of interest to B cells found in non-Hodgkin lymphomas (NHLs).

It has been shown that antibodies against CD79b block B-cell proliferation induced via the B-cell receptor, CD40, CD180, and chondroitin sulfate, but not via TLR4 or TLR9 (Brühl et al. 2015).

Anti CD20 antibody therapy is still the standard treatment for many patients suffering from B cell malignancies. Around two-thirds of patients benefit from this approach, which however means that one third does not.

On that background CD79b seems to offer new treatment options. In a clinical phase III trial reported by Tilly et al (2021), which involved 879 patients between the ages of 18 and 80 who had diffuse large B-cell lymphoma with an intermediate to high risk of an unfavourable outcome, patients received either standard therapy with anti-CD20 antibody plus chemotherapy (R-CHOP) or an antibody-drug conjugate with an antibody to CD79b and chemotherapy (pola-R-CHP). Significantly more patients in the pola-R-CHP group were still alive without the disease having progressed further. Patients treated with the antibody-drug conjugate and chemotherapy had a lower risk of disease progression, relapse and dying from the lymphoma.

The antibody drug conjugate used in this study is Polatuzumab vedotin (Polivy®), also known as DCDS4501A or RG7596.

Polivy® is an antibody drug conjugate comprising an IgG-shaped anti CD79b antibody and the toxin MMAE conjugated thereto via a linker/spacer consisting of para-aminobenzylcarbamate conjugated to a free thiol group of a cystein residue within a cystein engineered antibody (“Thiomab”), a cathepsin-cleavable linker comprising citrullin and valin, and an attachment group consisting of caproic acid and maleimide, to which the toxin is conjugated. Polivy® has an average drug-to-antibody ration (DAR) of 3.5 molecules of MMAE attached to each antibody, hence suffers from variability in DAR.

The antibody Polatuzumab is a humanized variant of the murine anti CD79b antibody SN8, described for the first time in 1993 by Okazaki et al. Polatuzumab and Polatuzumab vedotin (Polivy®) are disclosed inter alia in EP2176296 and U.S. Pat. No. 8,545,850.

It is one object of the present invention to provide new and improved options for cancer therapy.

These and other objects are solved by the features of the independent claims. The dependent claims disclose embodiments of the invention which may be preferred under particular circumstances. Likewise, the specification discloses further embodiments of the invention which may be preferred under particular circumstances.

### BRIEF DESCRIPTION OF THE FIGURES

FIGS. 1, 2 and 3 show results of in vitro cell viability assays of four recombinant immunotoxins according to the invention (IgG antibody fused via a G4S linker to the protein toxin anisoplin) and a benchmark antibody drug conjugate (Polatuzumab Vedotin (Polivy®)). It can be seen that the four immunotoxin according to the present invention have a much better IC₅₀ (in vitro efficacy) than the benchmark antibody drug conjugate.

FIGS. 4 and 5 show results of size exclusion chromatography experiments of recombinant immunotoxins according to the invention (IgG antibody fused via a G4S linker to the protein toxin anisoplin) and a benchmark recombinant immunotoxin (Polatuzumab fused via a G4S linker to the protein toxin anisoplin). FIG. 4A: ATB 704, FIG. 4B: ATB-580, FIG. 5A, column calibration, FIG. 5B: Benchmark (Polatuzumab). The recombinant immunotoxins according to the invention are highly stable, while the Polatuzumab-based benchmark (ATB-452) demonstrates higher aggregation propensity

FIGS. 6 and 7 show results of IHC staining on frozen human tissues from three (3) donors under conditions favoring high affinity interaction with a cognate receptor (on-target binding). Specific staining with the recombinant immunotoxins ATB-580, ATB-693, ATB-697 and ATB-704 (IgG antibody fused via a G4S linker to the protein toxin anisoplin) was seen only in CD79b-positive human tissues, such as lymph nodes, spleen and thymus. No off-target binding to CD79b-negative tissues was observed for all tested compounds.

FIG. 8 shows a comparison of anti-tumor activity in vivo of four recombinant immunotoxins according to the invention (IgG antibody fused via a G4S linker to the protein toxin anisoplin) and a benchmark recombinant immunotoxin (Polatuzumab fused via a G4S linker to the protein toxin anisoplin, called “ATB-747” herein), A s.c. CDX model in SCID mice (B-NHL representative cell line engrafted) was used for this purpose.

FIG. 9 shows an alignment of the different isoforms of CD79B, with an epitope as discussed in the specification in bold.

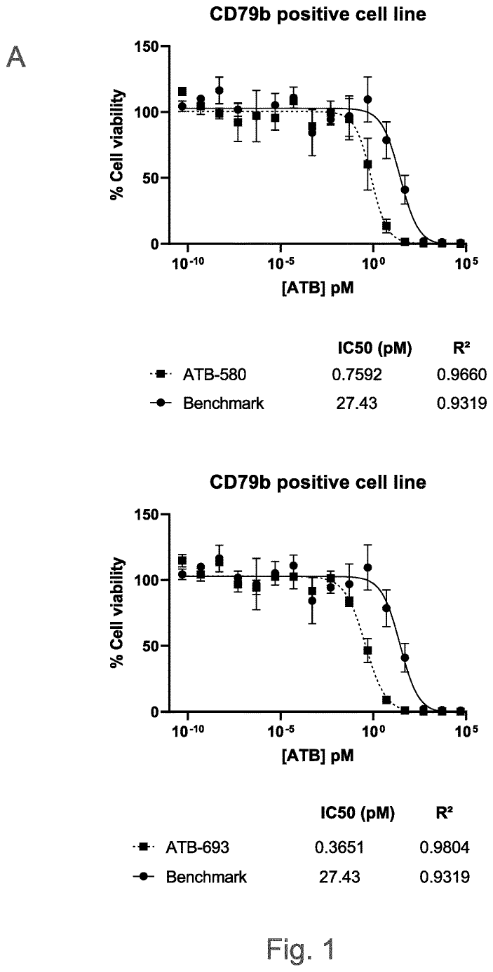

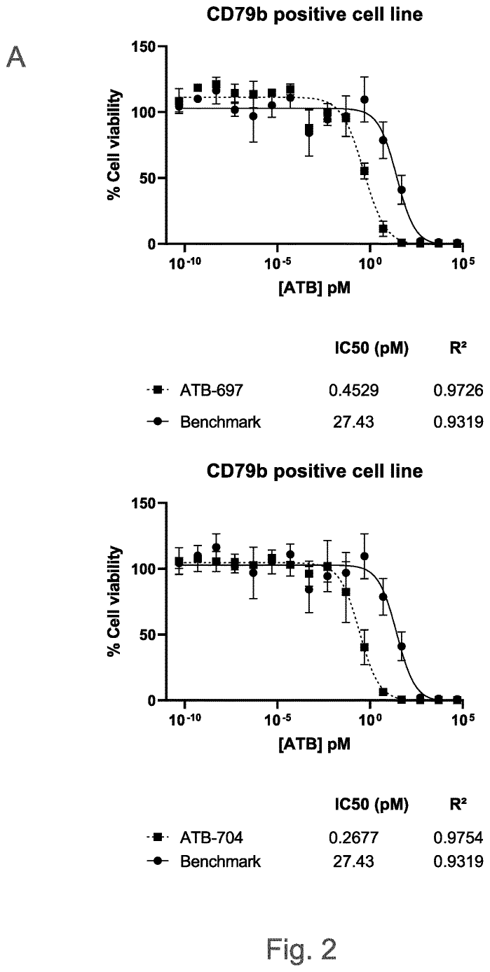

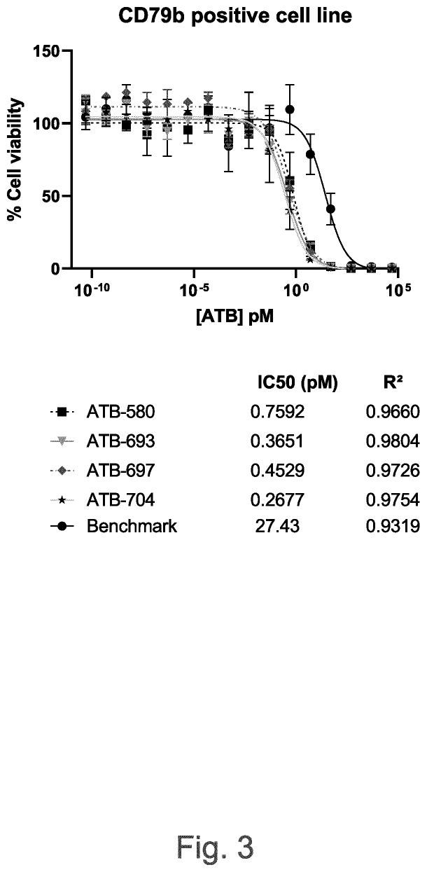

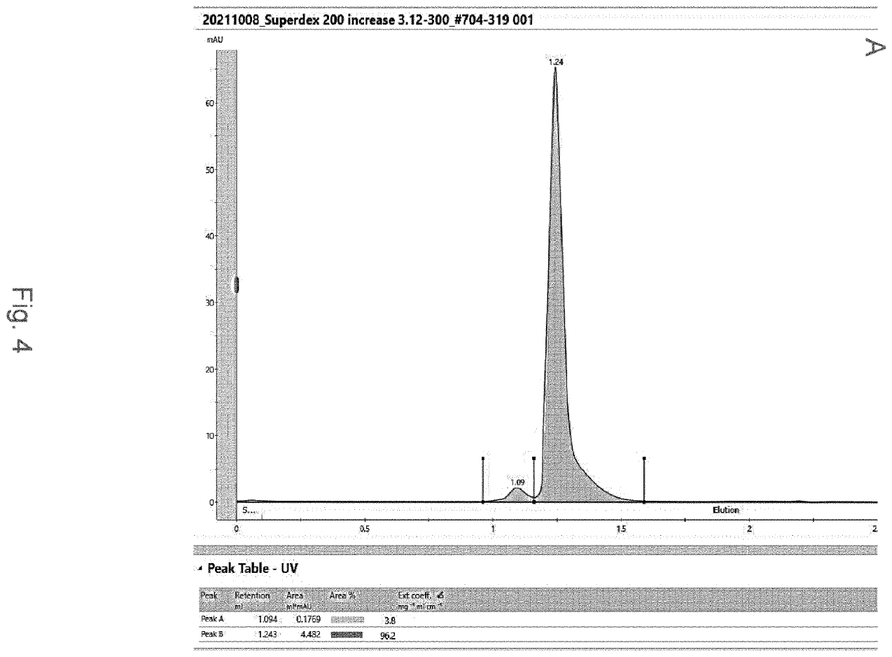

### DETAILED DESCRIPTION

According to one aspect of the invention, an antibody or a target-binding fragment or derivative thereof retaining target binding capacity to human CD79b is provided, which
a) comprises a set of six heavy chain/light chain complementarity determining regions (CDR) comprised in the heavy chain/light variable domain sequence pair selected from one of the following pairs of SEQ ID NOs:
   3 and 4,
   13 and 14,
   23 and 24, or
   33 and 34,
b) comprises a set of six heavy chain/light chain complementarity determining regions (CDR) selected from, in the order HCDR1; HCDR2; HCDR3; LCDR1; LCDR2 and LCDR3,
   i) SEQ ID NOs: 5, 6, 7, 8, 9 and 10
   ii) SEQ ID NOs: 15, 16, 17, 18, 19 and 20
   iii) SEQ ID NOs: 25, 26, 27, 28, 29 and 30, or
   iv) SEQ ID NOs: 35, 36, 37, 38, 39 and 40,
c) comprises a set of heavy chain/light chain complementarity determining regions (CDR) as set forth in option b), with the proviso that at least one of the CDRs has up to 3 amino acid substitutions relative to the CDRs comprised in the respective SEQ ID NOs, and/or
d) comprises a set of heavy chain/light chain complementarity determining regions (CDR) as set forth in option b) or c), with the proviso that at least one of the CDRs has a sequence identity of ≥66% to the CDRs comprised in the respective SEQ ID NOs,

The CDRs are embedded in a suitable protein framework so as to be capable to bind to human CD79b. The following table shows the antibodies according to the invention in condensed form.

TABLE 1
Antibody names and sequences used herein

| Antibody name | Sequences |
|---|---|
| ATB-580 | SEQ ID NO: 1-10 |
| ATB-693 | SEQ ID NO: 11-20 |
| ATB-697 | SEQ ID NO: 21-30 |
| ATB-704 | SEQ ID NO: 31-40 |

In one embodiment the antibody is a humanized antibody or fragment.

Methods for the production and/or selection of humanized mAbs are known in the art. For example, U.S. Pat. No. 6,331,415 by Genentech describes the production of chimeric antibodies, while U.S. Pat. No. 6,548,640 by Medical Research Council describes CDR grafting techniques and U.S. Pat. No. 5,859,205 by Celltech describes the production of humanized antibodies.

Humanized antibodies are antibodies in which the complementarity determining regions stem from a parent antibody taken from a non human species and are grafted into the framework (at least the variable domain) of a human antibody, like e.g. of an IgG1, IgG2 or IgG4. The humanized antibody binds the same target as the parent antibody, but, due to its grafting into a human framework, has reduced immunogenicity (like e.g. HAMA response). For this reason, a humanized antibody is structurally different from its parent (e.g. murine) antibody.

In humanization, the step of grafting the CDRs into a human framework is often followed by a step of affinity maturation, to reacquire affinity that was lost in the grafting process. This process further modifies the sequence of the human antibody, including its CDRs.

As used herein, the term “CDR” or “complementarity determining region” is intended to mean the non-contiguous antigen combining sites found within the variable region of both heavy and light chain polypeptides. These particular regions have been described inter alia by Kabat et al. (1977), Kabat et al. (1991 and Chothia et al. (1987), where the definitions include overlapping or subsets of amino acid residues when compared against each other.

Nevertheless, application of either definition to refer to a CDR of an antibody or grafted antibodies or variants thereof is intended to be within the scope of the term as defined and used herein.

Preferably, the CDRs as set forth in this specification are hence determined according to the Kabat or Chothia numbering set forth in table 2.

TABLE 2
CDR definitions

| | Kabat | Chothia |
|---|---|---|
| VH CDR1 | 31-35 | 26-32 |
| VH CDR2 | 50-65 | 53-55 |
| VH CDR3 | 95-102 | 96-101 |
| VL CDR1 | 24-34 | 26-32 |
| VL CDR2 | 50-56 | 50-52 |
| VL CDR3 | 89-97 | 91-96 |

As used herein, the term “framework” when used in reference to an antibody variable region is entered to mean all amino acid residues outside the CDR regions within the variable region of an antibody. Therefore, a variable region framework is between about 100-120 amino acids in length but is intended to reference only those amino acids outside of the CDRs.

In one embodiment, the term “capable to bind to target X” has to be understood as meaning that respective binding domain binds the target with a K_{D} of 10-4 or smaller. K_{D} is the equilibrium dissociation constant, a ratio of k_{off}/kₒₙ, between the antibody or fragment and its antigen. K_{D} and affinity are inversely related. The K_{D} value relates to the concentration of antibody or fragment (the amount of antibody or fragment needed for a particular experiment) and so the lower the K_{D} value (lower concentration) and thus the higher the affinity of the binding domain. The following table shows typical K_{D} ranges of monoclonal antibodies

TABLE 3
K_{D} and Molar Values

| K_{D} value | Molar range |
|---|---|
| 10⁻⁴ to 10⁻⁶ | Micromolar (μM) |
| 10⁻⁷ to 10⁻⁹ | Nanomolar (nM) |
| 10⁻¹⁰ to 10⁻¹² | Picomolar (pM) |
| 10⁻¹³ to 10⁻¹⁵ | Femtomolar (fM) |

Preferably, the antibody or fragment has up to 2 amino acid substitutions, and more preferably up to 1 amino acid substitution.

Preferably, at least one of the CDRs of the antibody or fragment has a sequence identity of ≥67%; ≥68%; ≥69%; ≥70%; ≥71%; ≥72%; ≥73%; ≥74%; ≥75%; ≥76%; ≥77%; ≥ 78%; ≥79%; ≥80%; ≥81%; ≥82%; ≥83%; ≥84%; ≥85%; ≥86%; ≥87%; ≥88%; ≥ 89%; ≥90%; ≥91%; ≥92%; ≥93%; ≥94%; ≥95%; ≥96%; ≥97%; ≥98%; ≥99%, and most preferably 100% to the respective SEQ ID NO.

“Percentage of sequence identity” as used herein, is determined by comparing two optimally aligned biosequences (amino acid sequences or polynucleotide sequences) over a comparison window, wherein the portion of the corresponding sequence in the comparison window may comprise additions or deletions (i.e., gaps) as compared to the reference sequence, which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same sequences. Two sequences are “substantially identical” if two sequences have a specified percentage of amino acid residues or nucleotides that are the same (i.e., at least 85%, 90%, 95%, 96%, 97%, 98% or 99% sequence identity over a specified region, or, when not specified, over the entire sequence of a reference sequence), when compared and aligned for maximum correspondence over a comparison window, or designated region as measured using one of the following sequence comparison algorithms or by manual alignment and visual inspection. The disclosure provides polypeptides that are substantially identical to the polypeptides exemplified herein. With respect to amino acid sequences, identity or substantial identity can exist over a region that is at least 5, 10, 15 or 20 amino acids in length, optionally at least about 25, 30, 35, 40, 50, 75 or 100 amino acids in length, optionally at least about 150, 200 or 250 amino acids in length, or over the full length of the reference sequence. With respect to shorter amino acid sequences, e.g., amino acid sequences of 20 or fewer amino acids, substantial identity exists when one or two amino acid residues are conservatively substituted, according to the conservative substitutions defined herein.

Preferably, at least one of the CDRs has been subject to CDR sequence modification, including
affinity maturation
reduction of immunogenicity

Affinity maturation in the process by which the affinity of a given antibody is increased in vitro. Like the natural counterpart, in vitro affinity maturation is based on the principles of mutation and selection. It has successfully been used to optimize antibodies, antibody fragments or other peptide molecules like antibody mimetics. Random mutations inside the CDRs are introduced using radiation, chemical mutagens or error-prone PCR. In addition, the genetic diversity can be increased by chain shuffling. Two or three rounds of mutation and selection using display methods like phage display usually results in antibody fragments with affinities in the low nanomolar range. For principles see Eylenstein et al. (2016) or US20050169925A1, the content of which is incorporated herein by reference for enablement purposes.

Engineered antibodies contain murine-sequence derived CDR regions that have been engrafted, along with any necessary framework back-mutations, into sequence-derived V regions. Hence, the CDRs themselves can cause immunogenic reactions when the humanized antibody is administered to a patient. Methods of reducing immunogenicity caused by CDRs are disclosed in Harding et al. (2010), or US2014227251A1, the content of which is incorporated herein by reference for enablement purposes.

According to one embodiment of the invention, the antibody or fragment comprises
a) the heavy chain/light chain variable domain (HCVD/LCVD) pairs set forth in the following pairs of SEQ ID NOs:
   3 and 4,
   13 and 14,
   23 and 24, or
   33 and 34,
b) the heavy chain/light chain variable domains (HCVD/LCVD) pairs of a), with the proviso that
   the HCVD has a sequence identity of ≥80% to the HCVD comprised in the respective SEQ ID NO, and/or
   the LCVD has a sequence identity of ≥80% to the LCVD comprised in the respective SEQ ID NO,
c) the heavy chain/light chain variable domains (HCVD/LCVD) pairs as set forth in option a) or b), with the proviso that at least one of the HCVD or LCVD has up to 10 amino acid substitutions relative to the HCVD or LCVD comprised in the respective SEQ ID NO,

It is provided that said antibody or fragment is capable to bind to human CD79b.

A “variable domain” when used in reference to an antibody or a heavy or light chain thereof is intended to mean the portion of an antibody which confers antigen binding onto the molecule and which is not the constant region. The term is intended to include functional fragments thereof which maintain some of all of the binding function of the whole variable region. Variable region binding fragments include, for example, functional fragments such as Fab, F(ab)₂, Fv, single chain Fv (scfv) and the like. Such functional fragments are well known to those skilled in the art. Accordingly, the use of these terms in describing functional fragments of a heteromeric variable region is intended to correspond to the definitions well known to those skilled in the art. Such terms are described in, for example, Huston et al., (1993) or Plückthun and Skerra (1990).

Preferably, the HCVD and/or LCVD has a sequence identity of ≥81%; ≥82%; ≥83%; ≥84%; ≥85%; ≥86%; ≥87%; ≥88%; ≥89%; ≥90%; ≥91%; ≥92%; ≥93%; ≥94%; ≥95%; ≥96%; ≥97%; ≥98%; ≥99%; or most preferably 100% to the respective SEQ ID NO.

According to one embodiment of the invention, at least one amino acid substitution is a conservative amino acid substitution.

A “conservative amino acid substitution”, as used herein, has a smaller effect on antibody function than a non-conservative substitution. Although there are many ways to classify amino acids, they are often sorted into six main groups on the basis of their structure and the general chemical characteristics of their R groups.

In some embodiments, a “conservative amino acid substitution” is one in which the amino acid residue is replaced with an amino acid residue having a similar side chain. For example, families of amino acid residues having similar side chains have been defined in the art. These families include amino acids with
basic side chains (e.g., lysine, arginine, histidine),
acidic side chains (e.g., aspartic acid, glutamic acid),
uncharged polar side chains (e.g., glycine, asparagine, glutamine, serine, threonine, tyrosine, cysteine),
nonpolar side chains (e.g., alanine, valine, leucine, isoleucine, proline, phenylalanine, methionine, tryptophan),
beta-branched side chains (e.g., threonine, valine, isoleucine) and
aromatic side chains (e.g., tyrosine, phenylalanine, tryptophan, histidine).

Other conserved amino acid substitutions can also occur across amino acid side chain families, such as when substituting an asparagine for aspartic acid in order to modify the charge of a peptide. Conservative changes can further include substitution of chemically homologous non-natural amino acids (i.e. a synthetic non-natural hydrophobic amino acid in place of leucine, a synthetic non-natural aromatic amino acid in place of tryptophan).

According to one embodiment of the invention, the human CD70b to which the antibody or fragment binds comprises
a) the amino acid sequence set forth in any one of SEQ ID NO: 41-44, or
b) an amino acid sequence that has at least 80% sequence identity with SEQ ID NO: 41-44, with the proviso that said sequence maintains CD79b activity.

In some embodiments, human CD79b comprises an amino acid sequence that has ≥81%, preferably ≥82%, more preferably ≥83%, ≥84%, ≥85%, ≥86%, ≥87%, ≥88%, ≥89%, ≥90%, ≥91%, ≥92%, ≥93%, ≥94%, ≥95%, ≥96%, ≥97%, ≥98 or most preferably ≥99% sequence identity with SEQ ID NO: 41-44

SEQ ID NO: 41 represents the amino acid sequence of human CD79b, accessible under NCBI reference. Generally, different variants and isoforms of CD79b exist, disclosed herein as SEQ ID NOs: 42-44. Likewise, mutants comprising conservative or silent amino acid substitutions exist, or may exist, which maintain full or at least substantial CD79b activity. These isoforms, variants and mutants are encompassed by the identity range specified above, meaning however that dysfunctional, non-active variants and mutants are excluded.

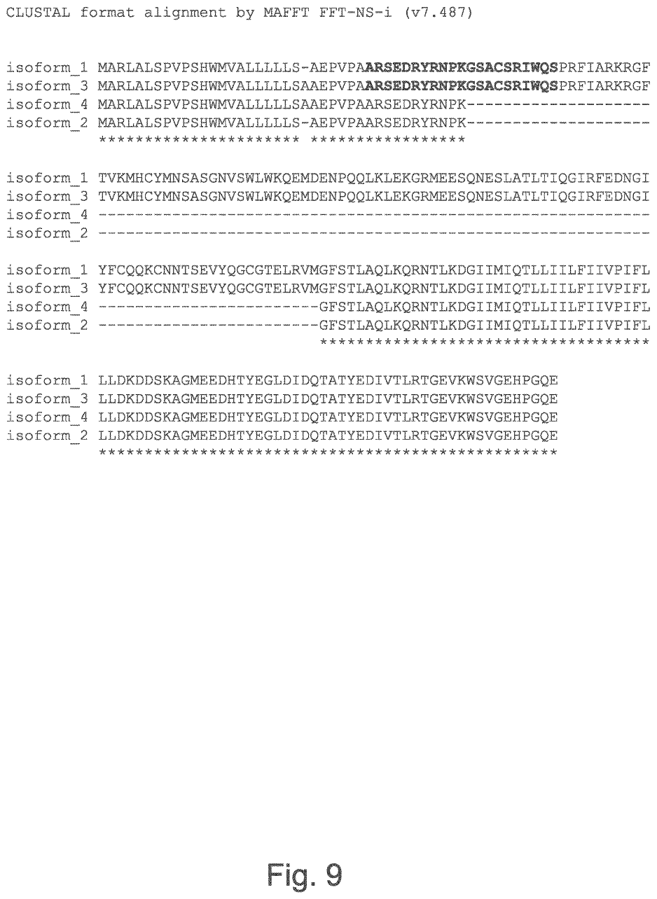

In this context, the inventors have surprisingly found that the antibodies according to the present invention bind all four isoforms of CD79b as referred to above. This is surprising because existing anti CD79b antibodies like SN8, recognize the epitope ARSEDRYRNPKGSACSRIWQS (SEQ ID NO: 61) which is present only in isoforms 1 and 3, while missing in isoforms 2 and 4 (see FIG. 9 for an alignment of the different isotypes). On that basis, the antibodies according to the invention have a surprisingly broad target spectrum According to one embodiment of the invention, the antibody or fragment is a monoclonal antibody, or a target-binding fragment or derivative thereof retaining target binding capacity to human CD79b.

According to one embodiment of the invention, the antibody or fragment is in at least one of the formats selected from the group consisting of: IgG, scFv, Fab, or (Fab)₂.

As used herein, the term “monoclonal antibody (mAb)” shall refer to an antibody composition having a homogenous antibody population, i.e., a homogeneous population consisting of a whole immunoglobulin, or a fragment or derivative thereof retaining target binding capacities.

Particularly preferred, such antibody is an IgG antibody, or a fragment or derivative thereof retaining target binding capacities. Immunoglobulin G (IgG) is a type of antibody. Representing approximately 75% of serum antibodies in humans, IgG is the most common type of antibody found in blood circulation. IgG molecules are created and released by plasma B cells. Each IgG has two antigen binding sites.

IgG antibodies are large molecules with a molecular weight of about 150 kDa made of four peptide chains. It contains two identical class y heavy chains of about 50 kDa and two identical light chains of about 25 kDa, thus a tetrameric quaternary structure. The two heavy chains are linked to each other and to a light chain each by disulfide bonds. The resulting tetramer has two identical halves, which together form the Y-like shape. Each end of the fork contains an identical antigen binding site. The Fc regions of IgGs bear a highly conserved N-glycosylation site. The N-glycans attached to this site are predominantly core-fucosylated diantennary structures of the complex type. In addition, small amounts of these N-glycans also bear bisecting GlcNAc and α-2,6-linked sialic acid residues.

According to one embodiment of the invention, the antibody is in at least one of the formats selected from the group consisting of IgG1, IgG2 or IgG4.

As used herein, the term “fragment” shall refer to fragments of such antibody retaining target binding capacities, e.g.
a CDR (complementarity determining region)
a hypervariable region,
a variable domain (Fv)
an IgG or IgM heavy chain (consisting of VH, CHI, hinge, CH2 and CH3 regions)
an IgG or IgM light chain (consisting of VL and CL regions), and/or
a Fab and/or F(ab)₂.

As used herein, the term “derivative” shall refer to protein constructs being structurally different from, but still having some structural relationship to, the common antibody concept, e.g., scFv, Fab and/or F(ab)₂, as well as bi-, tri- or higher specific antibody constructs, and further retaining target binding capacities. All these items are explained below.

Other antibody derivatives known to the skilled person are Diabodies, Camelid Antibodies, Nanobodies® (also known as single-domain antibodies), Domain Antibodies, bivalent homodimers with two chains consisting of scFvs, IgAs (two IgG structures joined by a J chain and a secretory component), shark antibodies, antibodies consisting of new world primate framework plus non-new world primate CDR, dimerized constructs comprising CH3+VL+VH, and antibody conjugates (e.g. antibody or fragments or derivatives linked to a toxin, a cytokine, a radioisotope or a label). These types are well described in the literature and can be used by the skilled person on the basis of the present disclosure, without adding further inventive activity.

Methods for the production of a hybridoma cell are disclosed in Köhler & Milstein (1975).

Methods for the production and/or selection of fully human mAbs are known in the art. These can involve the use of a transgenic animal which is immunized with the respective protein or peptide, or the use of a suitable display technique, like yeast display, phage display, B-cell display or ribosome display, where antibodies from a library are screened against human CD79b in a stationary phase.

In vitro antibody libraries are, among others, disclosed in U.S. Pat. No. 6,300,064 by MorphoSys and U.S. Pat. No. 6,248,516 by MRC/Scripps/Stratagene. Phage Display techniques are for example disclosed in U.S. Pat. No. 5,223,409 by Dyax. Transgenic mammal platforms are for example described in EP1480515A2 by TaconicArtemis.

IgG, IgM, scFv, Fab and/or F(ab)₂ are antibody formats well known to the skilled person. Related enabling techniques are available from the respective textbooks.

As used herein, the term “Fab” relates to an IgG/IgM fragment comprising the antigen binding region, said fragment being composed of one constant and one variable domain from each heavy and light chain of the antibody

As used herein, the term “F(ab)₂” relates to an IgG/IgM fragment consisting of two Fab fragments connected to one another by disulfide bonds.

As used herein, the term “scFv” relates to a single-chain variable fragment being a fusion of the variable regions of the heavy and light chains of immunoglobulins, linked together with a short linker, usually serine(S) or glycine (G). This chimeric molecule retains the specificity of the original immunoglobulin, despite removal of the constant regions and the introduction of a linker peptide.

Modified antibody formats are for example bi-or trispecific antibody constructs, antibody-based fusion proteins, immunoconjugates and the like. These types are well described in the literature and can be used by the skilled person on the basis of the present disclosure, with adding further inventive activity.

In one or more embodiments, the antibody or fragment is an isolated antibody, or a target-binding fragment or derivative thereof retaining target binding capacities, or an isolated antibody mimetic

In one or more embodiments, the antibody is an engineered or recombinant antibody, or a target binding fragment or derivative thereof retaining target binding capacities, or an engineered or recombinant antibody mimetic.

According to one embodiment of the invention, the antibody or fragment is an antibody in at least one of the formats selected from the group consisting of: IgG, scFv, Fab, or (Fab)₂.

According to another aspect of the invention, a nucleic acid is provided that encodes for at least one chain of the binding agent according to the above description.

In one embodiment, a nucleic acid, or a pair of nucleic acids, is provided which encodes for the heavy chain and the light chain, respectively, of the binding agent, in case the latter is a monoclonal antibody having a heteromeric structure of at least one light chain and one heavy chain.

Such nucleic acid can be used for recombinant production of the antibody or fragment or derivative thereof in a suitable expression system, like e.g. CHO cells or Nicotinia.

Such nucleic acid can also be used for pharmaceutic purposes. The nucleic acid can be an RNA molecule, or an RNA derivative comprising, e.g., modified nucleotides, like pseudouridine (Y) or N-1 Methyl Pseudouridine (m 14′) to provide stability and reduce immunogenicity (see, e.g., U.S. Pat. Nos. 8,278,036 and 9,428,535, the contents of which are incorporated herein for enablement purposes). In another embodiment, the RNA comprises the most GC-rich codon is selected to provide stability and reduce immunogenicity (see e.g. EP1392341 the content of which is incorporated herein for enablement purposes). The mRNA can for example be delivered in suitable liposomes and comprises either specific sequences or modified uridine nucleosides to avoid immune responses and/or improve folding and translation efficiency, sometimes comprising cap modifications at the 5′- and/or 3′ terminus to target them to specific cell types.

The nucleic acid can likewise be a DNA molecule. In such case, the molecule can be a cDNA that is optionally integrated into a suitable vector, e.g., an attenuated, non pathogenic virus, or is provided as one or more plasmids. Such plasmids can for example be administered to a patient by means of an electroporation device as e.g. disclosed in patent EP3397337B1, the content of which is incorporated herein for enablement purposes.

Generally, due to the degeneracy of the genetic code, there is a large number of different nucleic acids that have the capacity to encode for such chain. The skilled person is perfectly able to determine if a given nucleic acid satisfies the above criterion. On the other hand, the skilled person is perfectly able to reverse engineer, from a given amino acid sequence, based on codon usage tables, a suitable nucleic acid encoding therefore. For this purpose, software tools such as “reverse translate” provided by the online tool “sequence manipulation suite”, can be used. Hence, there are plenty of alternative DNA and RNA sequences that encode for the protein sequences as claimed. These alternative sequences shall be deemed to fall under the scope of the present invention.

According to another aspect of the invention, a recombinant immunotoxin, an immunocytokine, an antibody drug conjugate or an antibody-radionucleide conjugate is provided. This embodiment comprises the antibody or fragment according to the above description.

As used herein, the term “recombinant immunotoxin” relates to a fusion construct which comprises at least (i) one antibody or fragment according to the above description and (ii) a protein toxin or protoxin fused thereto. Such recombinant recombinant immunotoxin can be produced in suitable recombinant expression systems without subsequent need to conjugate the toxin to the antibody or fragment.

As used herein, the term “protein toxin” or “protein protoxin” is meant to encompass cytotoxic and/or cytostatic proteins. or the pro-variants thereof.

As used herein the term “cytostatic protein” refers to a protein that can inhibit cell proliferation or cell division without necessarily killing the cell. Suitably, the cytostatic agent inhibits the proliferation of tumor cells.

As used herein, the term “cytotoxic protein” refers to a protein that is harmful to cells and ultimately causes cell death. In some embodiments, the cytotoxic protein harms rapidly dividing cells such as tumor cells and causes tumor cell death, especially tumor cell death while not causing damage to or causing less damage to non-tumor cells.

The terms “protein toxin” or “protein protoxin”, refer without limitation to toxins that are, by their chemical nature, proteins (i.e., peptides having a length of ≥50 amino acid residues) or polypeptides (i.e., peptides having a length of ≥10-≤50 amino acid residues). A protoxin, in the meaning of the present invention, is a precursor of a toxin, also called a latent toxin, which needs to be activated, e.g., by cleaving off an inhibitory amino acid sequence, or by undergoing a conformational change. The terms “protoxin” and “protein protoxin” are used interchangeably here and mean the same subject matter.

Such protein toxin or protoxin can be selected from the group consisting of ribotoxins, endoribonucleases (RNases), Ribosome Inactivating Protein (RIP) and AB toxins.

The term “ribotoxin”, as used herein, relates to a group of extracellular ribonucleases secreted by fungi. Their most notable characteristic is their extraordinary specificity. They inactivate ribosomes by cutting a single phosphodiester bond of the rRNA that is found in a universally conserved sequence. This cleavage leads to cell death by apoptosis. However, since they are extracellular proteins, they must first enter the cells that constitute their target to exert their cytotoxic action. This entry constitutes the rate-determining step of their action.

Ribotoxins have been detected in many different fungi, including entomopathogenic and edible species, but the three-dimensional structure has only been resolved for three of them: α-sarcin (SEQ ID NO: 56, and deimmunized variant SEQ ID NO: 57), restrictocin, and hirsutellin A (HtA, SEQ ID NO: 48). The first two, produced by Aspergillus giganteus and Aspergillus restrictus, respectively, are nearly identical. HtA produced by the entomopathogenic fungus Hirsutella thompsonii, is much smaller and only shows 25% sequence identity with the other larger ribotoxins. Even so, it retains all the functional characteristics of the family. A second ribotoxin similar to HtA, anisoplin (SEQ ID NO: 49, and analogues and deimmunized variants thereof, disclosed herein as SEQ ID NOs: 50, 52, 53 and 54) is known (70% sequence identity to HtA). It is produced by the fungus Metarhizium anisopliae, another insect pathogen. Other ribotoxins that can be used in the context of the present invention are Angiogenin (SEQ ID NO: 51) and Ageritin (SEQ ID NO: 55).

The term “RNase”, as used herein, relates to a group of nucleases that catalyze the degradation of RNA into smaller components (“Ribonucleases”). In the meaning of the present invention, Ribonucleases act as endoribonucleases. In some embodiments, the RNase is one selected from the following group:
Onconase® (also known as ranpirnase or frog rnase): Different variants of Onconase®: exist, examples of which are published under the Uni Prot identifiers Q8UVX5, Q918V8, Q6EUW9, Q6EUW8, Q6EUW7 or P22069. While some examples in the present application use Q8UVX5 other Onconase variants can likewise be used.
RNase 1: Pancreatic ribonuclease (e.g. hRNase1, e.g. Uniprot identifier P07998 (SEQ ID NO: 58)
RNase 5: Angiogenin (e.g. hRNase 5, e.g. Uniprot identifier P03950)
RNase 2: Non-secretory ribonuclease (e.g. hRNase2, e.g. Uniprot identifier P10153)
RNase 3: Eosinophil cationic protein (e.g. hRNase3/Drosha, e.g. Uniprot identifier Q9NRR4 or P12724).
RNase 4: Ribonuclease 4 (e.g. hRNase4, e.g. Uniprot identifier P34096)
RNase 6: Ribonuclease K6/Ribonuclease T2/Ribonuclease K3 (e.g. hRNase6, e.g. Uniprot identifier Q93091)
RNase 7: Ribonuclease 7/Ribonuclease A E1 (e.g. hRNase7, e.g. Uniprot identifier Q9H1E1)
RNase 8: Ribonuclease 8 (e.g. hRNase8, e.g. Uniprot identifier Q8TDE3)

The above Uniprot identifiers have exemplary purpose only. Other variants can likewise be used. The skilled person can find such variants with routine efforts in the respective databases.

Ribosome-inactivating proteins (RIPs) are toxic N-glycosidases that depurinate eukaryotic and prokaryotic rRNAs, thereby arresting protein synthesis during translation. RIPs are widely present in various plant species and within different tissues. Those protein are known to play a key role in defense against pathogens and have been suggested to confer disease resistance. Plant based RIPs have so far been found in more than 50 different species from 14 families, including the Cucurbitaceae, Euphorbiaceae, Poaceae and Caryophyllales. In addition to plant, RIP have also been found in bacteria's, fungi, algae and even in mosquitoes.

RIPs constitute a large family of proteins that can be classified following their structural composition, RIP type I and type II.

Type I RIPs share a low molecular weight around 30 K_{D}, resulting as single-chain proteins. The single-chain of type I RIPs consists of an enzymatically active domain (A domain or alpha domain) exerting N-glycosidase activity.

Type II RIPs are larger protein with a weight comprised between 50-65 kDa, characterized by an enzymatically active A-chain and a slightly larger B chain (or beta chain, a lectin subunit) with galactose-like sugars.

In addition to RIPs type I and II, a third class-type III)—is reported with few members bearing N-terminal domain which is correlative to the A domain of RIPs and fused to an unknown functional C-terminal domain.

According to several embodiments, the Ribosome-inactivating protein (RIP) is at least one selected from the group consisting of:
Momordin.
Bryodin I (SEQ ID NO: 62)
Cucurmosin
Bryodin II (SEQ ID NO: 63)
Trichosanthin
Karasurin
MOMC
ME1, and/or.
ME2

AB toxins are two-component protein complexes secreted by a number of pathogenic bacteria. They can be classified as Type III toxins because they interfere with internal cell function. They are named AB toxins due to their components: the “A” component is usually the “active” portion, and the “B” component is usually the “binding” portion. The “A” subunit possesses enzyme activity, and is transferred to the host cell following a conformational change in the membrane-bound transport “B” subunit. These proteins consist of two independent polypeptides, which correspond to the A/B subunit moieties. The enzyme component (A) enters the cell through endosomes produced by the oligomeric binding/translocation protein (B), and prevents actin polymerisation through ADP-ribosylation of monomeric G-actin.

Examples of the “A” component of an AB toxin include C. perfringens iota toxin Ia, C. botulinum C2 toxin CI, and Clostridium difficile ADP-ribosyltransferase. Other homologous proteins have been found in Clostridium spiroforme.

An example of the B component of an AB toxin is Bacillus anthracis protective antigen (PA) protein, B. anthracis secretes three toxin factors: the protective antigen (PA); the oedema factor (EF); and the lethal factor (LF). Each is a thermolabile protein of ˜80 kDa. PA forms the “B” part of the exotoxin and allows passage of the “A” moiety (consisting of EF or LF) into target cells. PA protein forms the central part of the complete anthrax toxin, and translocates the A moiety into host cells after assembling as a heptamer in the membrane.

The Diphtheria toxin also is an AB toxin. It inhibits protein synthesis in the host cell through phosphorylation of the eukaryotic elongation factor 2, which is an essential component for protein synthesis. The exotoxin A of Pseudomonas aeruginosa is another example of an AB toxin that targets the eukaryotic elongation factor 2.

The AB5 toxins are usually considered a type of AB toxin, characterized by B pentamers. Less commonly, the term “AB toxin” is used to emphasize the monomeric character of the B component.

The two-phase mechanism of action of AB toxins is of particular interest in cancer therapy research. The general idea is to modify the B component of existing toxins to selectively bind to malignant cells. This approach combines results from cancer immunotherapy with the high toxicity of AB toxins, giving rise to a new class of chimeric protein drugs, called immunotoxins.

In one embodiment, the recombinant immunotoxin comprises
i) an antibody according to the above description,
ii) a toxin according to the above description and
iii) a peptide linker connecting the two elements i) and ii).

Linkers suitable for this purpose are disclosed herein as SEQ ID NOs: 45, 46, 47 and 60.

In one embodiment, the recombinant immunotoxin comprises the antibody 580, defined by the amino acid sequences SEQ ID NOs: 3-10 (SEQ ID NOs: 3 and 4: variable domains, SEQ ID NOs: 5-10: CDRs).

In one embodiment, the recombinant immunotoxin comprises the antibody 704 defined by the amino acid sequences SEQ ID NOs: 33-40 (SEQ ID NOs: 33 and 34: variable domains, SEQ ID NOs: 35-40: CDRs). In one embodiment, the recombinant immunotoxin comprises the ribotoxin aniosplin or an analogue (selected from any one of SEQ ID NOs: 49, 50, 52-54). In one embodiment, the recombinant immunotoxin comprises a peptide linker which is not cleavable by mammalian proteases, like e.g. a G4S linker (SEQ ID No: 60).

In one embodiment, the recombinant immunotoxin comprises
(i) the antibody 580, defined by the amino acid sequences SEQ ID NOs: 3-10.
(ii) the ribotoxin aniosplin having the sequence SEQ ID NO: 49 and
(iii) a peptide linker having the sequence SEQ ID NO: 60

In one embodiment, the recombinant immunotoxin comprises
(i) the antibody 704, defined by the amino acid sequences SEQ ID NOs: 33-40.
(ii) the ribotoxin aniosplin having the sequence SEQ ID NO: 49 and
(iii) a peptide linker having the sequence SEQ ID NO: 60

Preferably, the antibody in the recombinant immunotoxin is in the IgGI format.

As used herein, the term “immunocytokine” relates to a fusion construct which comprises at least (i) one antibody or fragment according to the above description and (ii) an immunomodulatory cytokine fused thereto. Such recombinant recombinant immunocytokine can be produced in suitable recombinant expression systems without subsequent need to conjugate the cytokine to the antibody or fragment.

Immunocytokines can be used to improve site-specific delivery and prolong the cytokine half-life. Immunocytokines are delivered systemically but can specifically target via specific tumor antigens. Suitable cytokines to be fused to the antibody are, inter alia, TNFalpha, IL2, IL12 and IL15. In such way, the maximum tolerated dose can be increased, and in IL 12 was found to be 30 times higher than the maximum tolerated dose of IL 12 alone.

As used herein, the term “antibody drug conjugate” relates to a construct comprising an antibody or fragment thereof to which a toxin is bound covalently. Said toxin is typically a small molecular toxin having a molecular weight of ≤2500 Da, and is oftentimes selected from the group consisting of
Maytansines
Monomethyl auristatine
Calicheamicins,
Doxorubicins
Pyrrolobenzodiazepine,
Methotrexate,
Topoisomerase 1 inhibitors
Glucocorticoid Receptor Modulators (GRM)
Taxanes
Anthracyclines,
Alpha-amanitin, and/o
Cyclosporines

Some examples encompass, but are not limited, to SN38, Exatecan, Dexamethasone, Budesonide, Mertansine, Ansamitocin, Ravtansin, DM4, DM1, Ozogamicin, Monomethyl Auristatin F (MMAF); Monomethyl Auristatin E (MMAE)

Contrary to immunotoxins and immuncytokines, the effector has to be conjugated to the antibody in a separate step. Typically, this is accomplished by using a linker/spacer consisting of para-aminobenzylcarbamate conjugated to a free thiol group of a cystein residue within the antibody, a cathepsin-cleavable linker comprising citrullin and valin, and an attachment group consisting of caproic acid and maleimide, to which the toxin is conjugated.

Here, issues of site specificity of the conjugation reaction and stochimetry between antibody and toxin play an important role. One attempt to solve these issues encompasses the so-called thiomab approach, in which the antibody is cysteine engineered to create preferred conjugation sites for the linker/spacer (see Panowski et al 2014, the content of which is incorporated herein by reference for enablement purposes).

Other attempts make use of specific enzymes that can be used to couple the toxin to the antibody in a stochiometric and site specific manner, like e.g. sortases or transglutaminases (see e.g., WO2014140317A1 and WO2020188061A1, the contents of which are incorporated herein by reference for enablement purposes).

Antibody-radionucleides comprise an antibody or fragment thereof that is labeled with at least one radionucleide, like e.g. Yttrium⁹⁰, Iodine¹³¹ or Lutetium¹⁷⁷. Such molecules are for example disclosed in Steiner & Neri 2011, the content of which is incorporated herein by reference for enablement purposes.

According to another aspect of the invention, a pharmaceutical composition is provided, the composition comprising the antibody or fragment according to the above description, the nucleic acid to the above description or the recombinant immunotoxin, immunocytokine, antibody drug conjugate or antibody-radionucleide to the above description, and optionally one or more pharmaceutically acceptable excipients.

According to another aspect of the invention, a combination is provided, comprising (i) the antibody or fragment according to the above description, the nucleic acid to the above description, the recombinant immunotoxin, immunocytokine, antibody drug conjugate or antibody-radionucleide to the above description, or the pharmaceutical composition to the above description and (ii) one or more therapeutically active compounds.

According to another aspect of the invention, the use of the antibody or fragment according to the above description, the nucleic acid to the above description, the recombinant immunotoxin, immunocytokine, antibody drug conjugate or antibody-radionucleide to the above description, the pharmaceutical composition to the above description or the combination to the above description is provided (for the manufacture of a medicament) in the treatment of a human or animal subject
being diagnosed for,
suffering from or
being at risk of developing
a neoplastic disease, or for the prevention of such condition.

This language is deemed to encompass both the swiss type claim language accepted in some countries (in this case, brackets are deemed absent) and EPC2000 language (in this case, brackets and content within the brackets is deemed absent).

According to another aspect of the invention, a method for treating or preventing a neoplastic disease is provided, which method comprises administration, to a human or animal subject, of the antibody or fragment according to the above description, the nucleic acid to the above description, the recombinant immunotoxin, immunocytokine, antibody drug conjugate or antibody-radionucleide to the above description, the pharmaceutical composition to the above description or the combination to the above description, in a therapeutically sufficient dose.

According to another aspect of the invention, a therapeutic kit of parts is provided comprising:
a) the antibody or fragment according to the above description, the nucleic acid to the above description, the recombinant immunotoxin, immunocytokine, antibody drug conjugate or antibody-radionucleide to the above description, the pharmaceutical composition to the above description or the combination to the above description,
b) an apparatus for administering the composition, composition or combination, and
c) instructions for use.

### Examples

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

All amino acid sequences disclosed herein are shown from N-terminus to C-terminus; all nucleic acid sequences disclosed herein are shown 5′-≥3′.

### Materials and Methods

Genetic construct of anti CD79B antibodies

Full length Rituximab HC and LC sequences have been used to develop mAb based binder-toxin fusion proteins. Variable parts sequences of the heavy and light chains of anti CD79B that have been developed by the inventors from humanization of murine antibody SN8 and fused to a human IgG1 Fc part sequence.

### Genetic Construct Comprising an Antibody Anti CD79B

Anti CD79B antibodies have been developed using hybrid humanization strategy on a murine antibody (SN8). Full length HC and LC antibody sequences have been used to develop antibody-based binder-toxin fusion proteins. A G4S sequence was then respectively used to fuse the Anispolin sequence at the C-terminal part of the HC to obtain HC-G4S-Anisoplin+LC Another binder-toxin fusion protein was realized with scFv-Fc, HC and LC part linked to Anisoplin or other ribotoxin with cleavage site to obtain, HC—FCS-Anisoplin+LC, HC—FCS-Anisoplin+LC and LC-FCS-Anisoplin+HC-FCS-Anisoplin. These sequences were produced by gene synthesis flanked with Xbal and Iscel.

Transient Expression in Nicotiana benthamiana Plant Leaves

Nicotiana benthaminana grown under 16h light/8h darkness photocycle, 22+/−3° C. 7-8 weeks old plants leaves were transiently transformed by syringe infiltration. Agrobacterium tumefaciens GV3101 (pMP90) harboring the undisclosed plasmid containing genetic construct reaching an 600 nm optical density (OD₆₀₀) around 0.8-1.0 were collected by centrifugation at 3500g for 10 min. Eventually, bacteria were adjusted to an OD₆₀₀ of 0.5 in infiltration buffer (10 mM MgCl2, 10 mM MES, 100 μM acetosyringone, pH 5,6) and the mixture was infiltrated using a needless syringe. Infiltrated regions were harvested 4 and 6 days post agroinfiltration. Entire leaves harvested 4 days post agroinfiltration were used for protein A purification.

Expression in N. tabacum Cells

Nicotiana tabacum plant suspension cells were grown 5 days at 130 rpm, 25° C. in plant culture media as described by Nagata et al. (1992), the content of which is incorporated herein. Agrobacterium tumefaciens LBA4404 (pBBR1MCS-5.virGN54D) harboring the pPZP-ATB binary plasmids reaching an 600 nm optical density (OD₆₀₀) around 0.8-1.0 were collected by centrifugation at 2000g for 5 min. Plant cells and bacterial cells were then cocultivated in cocultivation media for 30 min before a 2000g 5 min centrifugation. After supernatant removal, cells were plated on solid cocultivation media for two days. In the case of transient transformation, cells were then collected and washed three times and cultivated in plant cultivation media containing Cefotaxim and Carbeniclin before being harvested for further analysis. In the case of stable transformation, after the 2 days of solid cocultivation, cells were washed and plated on plant media containing selective kanamycin and Cefotaxim and Carbeniclin antibiotics. Callus were selected 4 weeks later and subcultured on solid media or in liquid suspension cultures for subsequent analysis.

### Protein a Purification

Four days post agroinfiltration, leaves were collected, weighted and grinded in a blender using 2 mL of extraction buffer (TRIS 0.1M, NaCl 460 mM, EDTA 5 mM, Sodium metabisulfite 5 mM pH 7.5per gram of fresh agroinfiltrated leaves. The mixture was then filtered through a double Miracloth (Millipore) layer. The filtrate was then centrifugated at 4° C. for 10 min at 40.000g. Supernatant was then loaded onto protein A resin preequilibrated with washing buffer. Resin was then washed with 10 column volume of 60 mM TRIS 25 mM, 460 mM NaCl pH 7.5. and elution was performed using 100 mM glycine, 460 mM NaCl pH3.0 directly buffered with 10% Tris 1M pH8.0. Enriched protein fractions were then collected, dialyzed and freeze in liquid nitrogen.

The purified binder-toxin proteins were visualized by SDS-PAGE.

### Size Exclusion Chromatography

After protein A, the enriched protein fraction is loaded on Sephacryl™ S-300 HR column (Cytiva). The main pic is collected and residual pic is removed from the collected fractions before pooling.

### Protein Analysis: SDS-PAGE and Westernblot

Proteins were boiled for 5 min in reducing or non-reducing SDS loading buffer (80 mM Tris-HCl, pH 6.8, 2% SDS, 10% glycerol, 0.005% bromophenol blue), centrifuged for 5 min at 13 000 rpm and separated by SDS-PAGE (4-20% polyacrylamide).

For Western blotting, proteins were electrotransferred onto a PVDF membrane (Biorad) using a semi-dry electrophoretic device (Biorad Trans-Blot Turbo); then, the membrane was blocked for 1 h at room temperature with 3% (w/v) non-fat milk powder in TBST buffer (50 mM Tris-HCl, 150 mM NaCl, 0.5% Tween® 20 (also known as Polysorbate 20), pH 7.5) and then incubated (TBS-Tween® 0.1%+0.5% non-fat dry milk) for 1 h at room temperature with HRP-conjugated antibodies against the anti-human IgG Fc specific region (A0170; Sigma-Aldrich), at a dilution of 1:10.000 or polyclonal against Anisoplin primary antibody from at a dilution of 1:50.000 (internal reference). The anti-human Fc antibody and the anti Anisoplin were followed by HRP-conjugated anti-rabbit antibodies (Synabs), at a dilution of 1:5 000. Proteins were detected by enhanced chemiluminescence (Amersham Imager 600/GE; GE Healthcare).

### Protein Analysis Analytical Size Exclusion Chromatography

Purified proteins were analyzed by size exclusion chromatorgraphy, using the Cytiva high resolution Superdex™ 200 Increase 10/300 GL column on an Äkta Pure™ or Äkta Go™ chromatography systems. The column was equilibrated with 2 column volumes of PBS at a flow rate of 1 mL/min. Purified proteins samples at a concentration range between 1 to 10 mg/mL were centrifugated for 5 minutes at 20000g. The supernatant was then harvested and 200 μL were loaded on a 100 μL capillary sample loop. The sample in the loop is then loaded on the column at a flow rate of 0.75 mL/min, and then eluted with 1.5 CV of PBS. The elution profile is monitored by absorbance (UV).

### In Vitro Cytotoxicity Assay

The effect of the binder toxin fusion proteins on the viability of cell lines expressing CD79b was assessed using the CellTiter-Glo® Assay (Promega, G9241). In this assay, mono-oxygenation of luciferin is catalyzed by luciferase in presence of Mg 2+ and ATP. This reaction generates a luminescent signal proportional to the number of viable cells.

Depending on the cell line tested, cells were seeded in the cavities of a 96-well plate at a density of 2000 or 5.000 cells/well in 50 μl of growth medium (RPMI1640). Serial dilutions of binder toxin fusion were prepared by adding 10 μl of binder toxin fusion or buffer (PBS, Tween® 0.02%) to 40 μl of growth medium. The mixture was added to the cells and incubated for 72 hours at 37° C. with 5% CO2. Binder toxin fusion were tested in duplicate. Buffer served as a negative control, medium and cells only served as blank and untreated control, respectively.

After 72 hours, plates were equilibrated at room temperature for 30 minutes and 100 μl of CellTiter-Glo® reagent were added to each well. The plates were subsequently placed on a shaking platform for 2 minutes then signal was allowed to stabilize for 10 minutes at room temperature in the dark. Luminescence was then recorded.

To determine the percentage of viability, the average luminescence signal of the blanks (growth medium only) was subtracted from each well and average luminescence signal of untreated cells was set as 100% viability. The average signal of treated cells was then normalized and plotted as a function of the binder toxin fusion concentration.

The anti-CD79b based binder-toxin fusion proteins were evaluated on B-cell lymphoma cells line (B-NHL, CD79+) and non-target cells K562 (CD79−).

### Immuno HistoChemistry-Tissue Micro Arrays (TMAs)

Binder toxin fusion have been labelled with fluorescein isothiocyanate FITC using an antibody labelling kit “Pierce™” from Thermo Scientific.

Slides containing CD79b positive cell line (B-NHL representative) and CD79b negative cells (K562) were used for method development and for defining optimal concentration of the antibodies for further studies. TMAs containing human (T6234701-1/2) normal frozen tissues were used for the cross-reactivity studies (Biochain Institute Incorp, CA USA).

Fixation is performed in cold acetone for 10 minutes and then air dried for 10 minutes. PBS+10% of normal human serum (Jackson Immunoresearch, 009-000-121) is added for 20 minutes. The normal human serum is removed and tissue blocks are incubated for 1 h with FITC labeled binder toxin fusion. The blocks are washed in PBS for 3 minutes before incubation with rabbit anti FITC (Serotec, #4510-7804) at dilution 1:1000 for 30 minutes.

Material is washed in PBS for 3 minutes before incubation with ready to use polymer BrightVision anti rabbit/HRP (Immunologic, DPVR110HRP) for 30 minutes. The tissue blocks are washed for 3 minutes in TRIS buffer (0.05M Trizmabase-Sigma, T1503-500 g—in distilled water). Block tissue are incubated 5 minutes in DAB (50 mg Diaminobenzidine-Sigma, #D5637-5G-dissolved in 100 ml Tris buffer (see above) with addition of 100 ml H2O2-Merck, 1.07209.0250-just prior to incubation).

Tissue blocks are washed for 3 minutes in distilled water and then counterstained in hematoxylin for 10 second before a quick wash in distilled water. Tissue blocks are dehydrated in 70-90-95% ethanol for 2 minutes each step then 2× 5 minutes in ethanol 99.5% and 3× 5 minutes in X-tra solve ((Medite, 41-5213-00). Section are prepared with cover slips and mounting media (Medite, 41-5219-00).

The staining was judged as negative (0), weak (1+), weak to moderate (1-2+), moderate (2+), moderate to strong (2-3+) or strong (3+).

### Cell Line Derived Xenograft

B-NHL representative cell line has been injected in peritoneum of CB17.SCID mice. When tumour reached a size around 1 cm³, CD79B expression has been confirmed by FACS analysis and tumours were sectioned in small fragments and transplanted in CB17.SCID mice. When mean tumour volume reached the desired size, animals were stratified in 6 groups of 7 animals with an average tumour volume of 0.139 cm3.

All binder toxin fusion used in vivo have been purified by SEC, removed from aggregates. Animals received a single dose of 20 mg/kg atbodies ATB-747, ATB-580, ATB-693, ATB-697 and ATB 704 or vehicle (PBS, Tween® 0.02%) via intravenous administration. Body weight and tumour volume were monitored three times per week.

Mean tumour volume for each group was plotted in function of days after treatment start. Tumour growth curves were plotted for individual animals of each group. Mice have been sacrificed when tumour volume get closer to 1.5 cm3 for ethical reason. GraphPad Prism 9.3.1 software was used to perform statistical analysis. To compare the different groups of the study, a two-way ANOVA as performed.

### Cell Viability Assay

Purified binder toxin fusions have been evaluated on cancer cell line for they cytotoxicity. All humanized anti CD79B toxin fusions (ATB-580-693-697-704) have shown to impair positive cell line viability. Moreover, we demonstrated superiority over a marketed ADC, Polivy® (Polatuzumab vedotin) that target the CD79B. Those results translate the superior mode of action of binder toxin fusion compared to antibody drug conjugated.

### OFF-TARGET/CROSS REACTIVITY ASSESSMENT

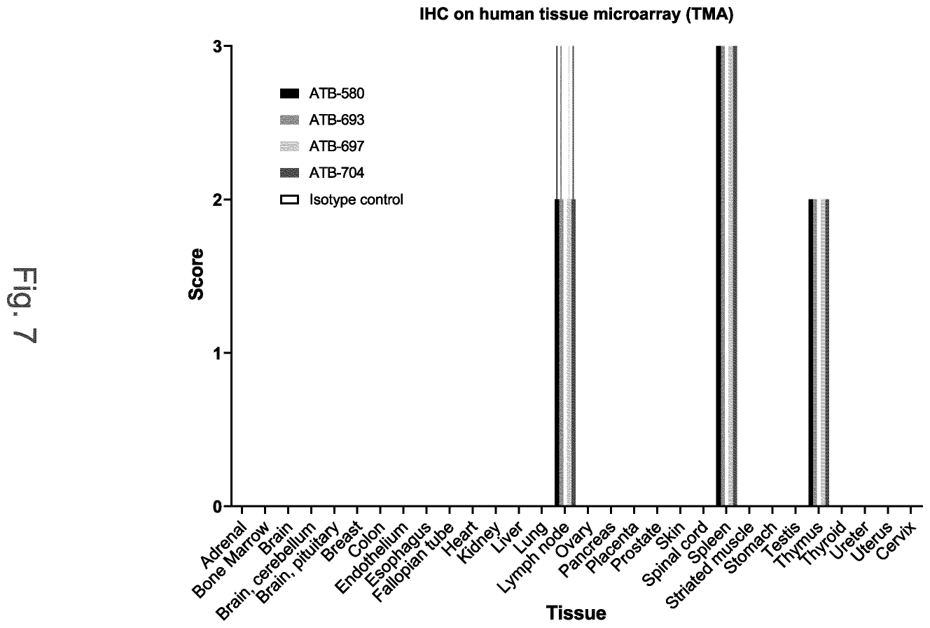

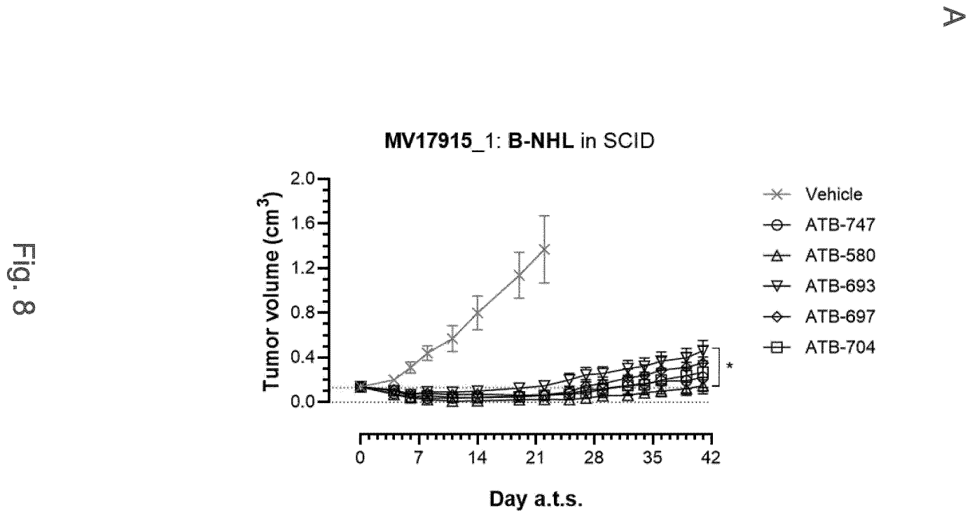

FIG. 7 demonstrated no off-target binding from all humanized anti CD79B toxin fusion (ATB-580-693-697-704). Only the tissues containing targeted B-cells are stained (lymph node— spleen-thymus). Comparable data have been obtained on cynomolgus and mice frozen tissues (data not shown).

### In Vivo Efficacy

### Polatuzumab

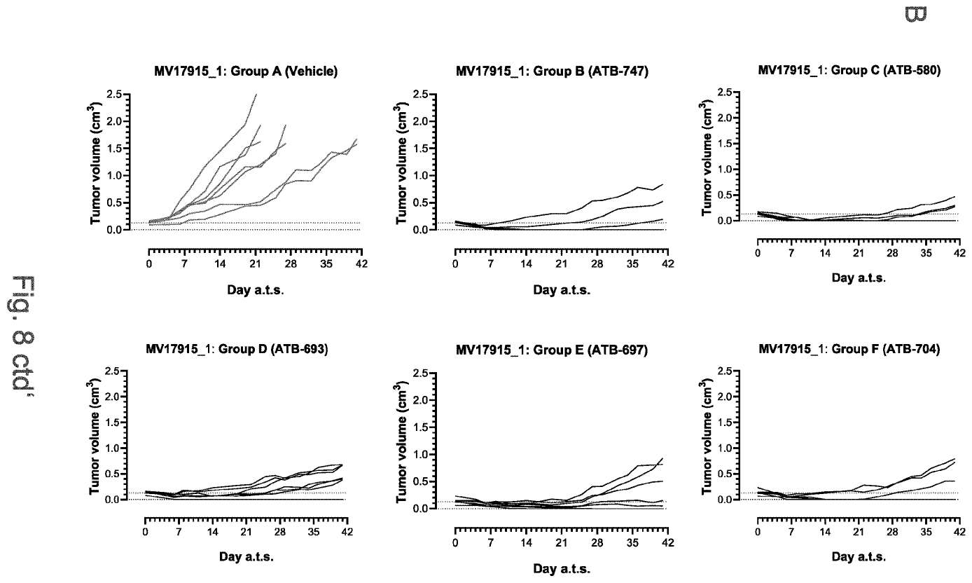

As illustrated in FIG. 8, all humanized recombinant anti CD79B immunotoxins (ATB-580-693-697-704) demonstrated a strong and long-standing anti-tumor effect on a CDX model. In particular, ATB-580 overperformed the benchmark polatuzumab recombinant immunotoxin (fused via a G4S linker to the protein toxin anisoplin) ATB-747 (FIG. 8).

### Analytical Size Exclusion Chromatography SEC

Purified proteins were analyzed by size exclusion chromatography, using the Cytiva high resolution Superdex™ 200 Increase 10/300 GL column on an Äkta Pure™ or Äkta Go™ chromatography systems. The column was equilibrated with 2 column volumes of PBS at a flow rate of 1 mL/min. Purified proteins samples at a concentration range between 1 to 10 mg/mL were centrifugated for 5 minutes at 20000g. The supernatant was then harvested and 200 μL were loaded on a 100 μL capillary sample loop. The sample in the loop is then loaded on the column at a flow rate of 0.75 mL/min, and then eluted with 1.5 CV of PBS. The elution profile is monitored by absorbance (UV).

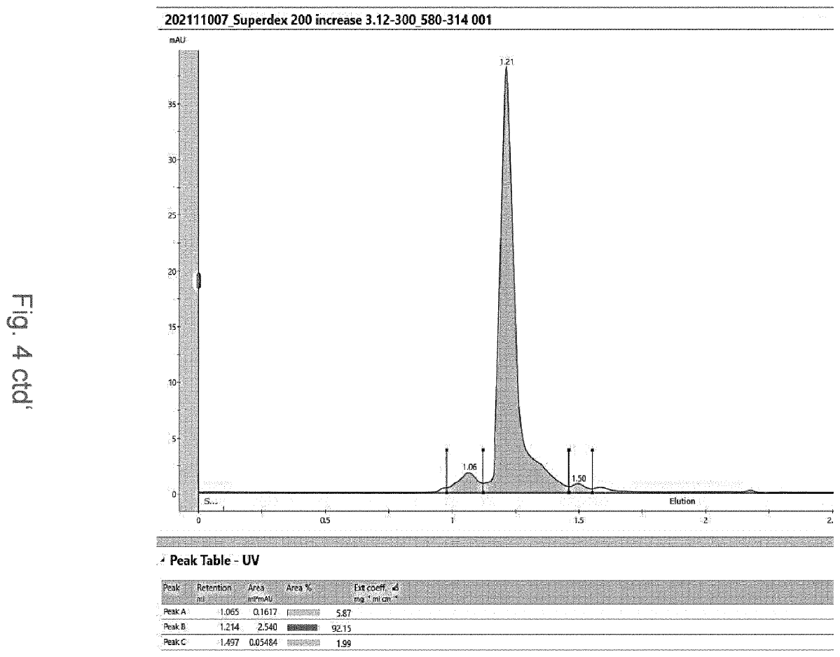

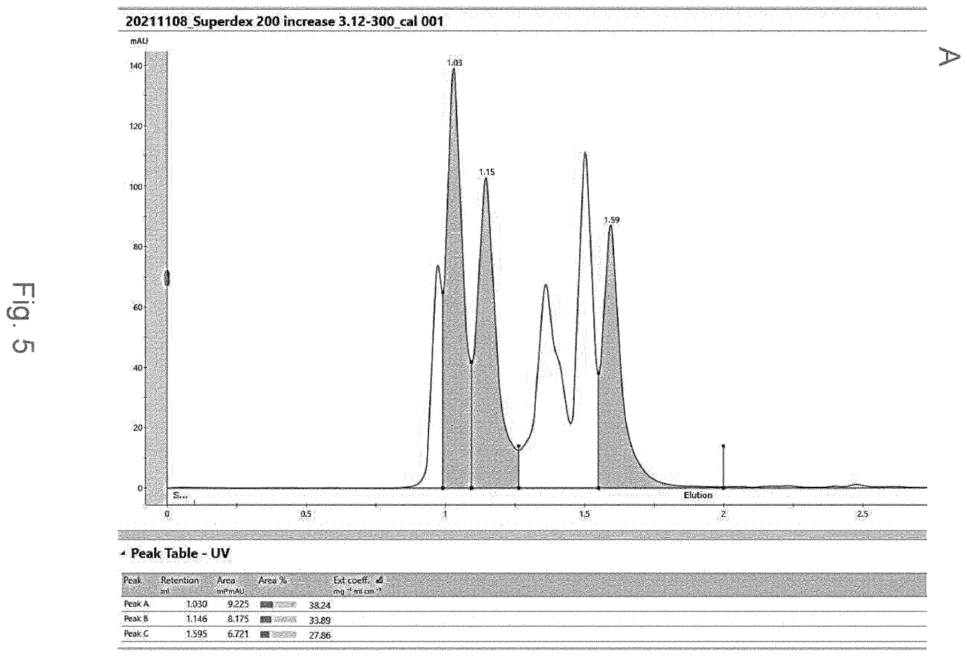

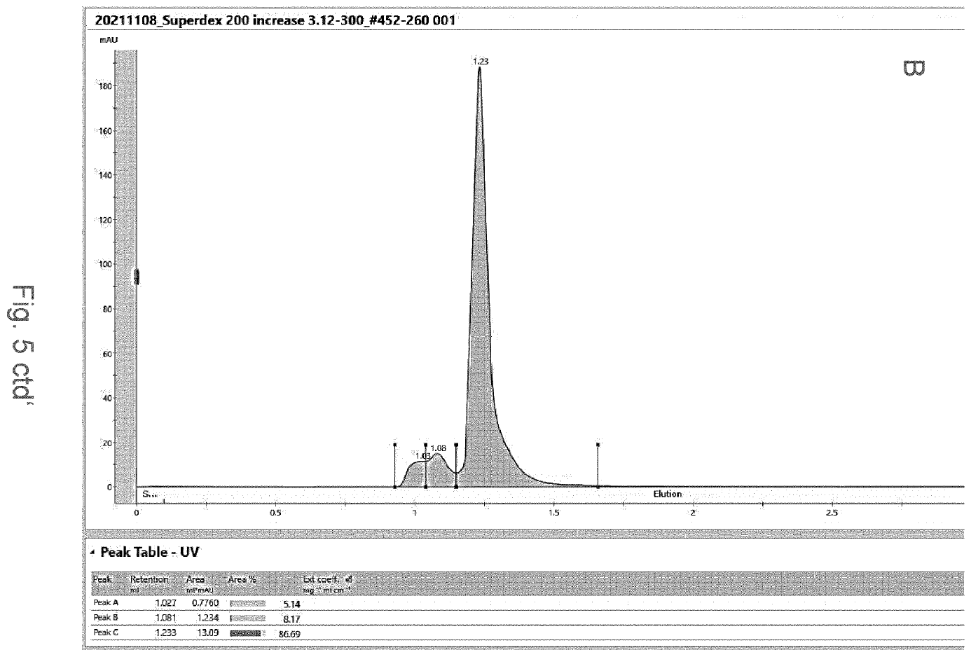

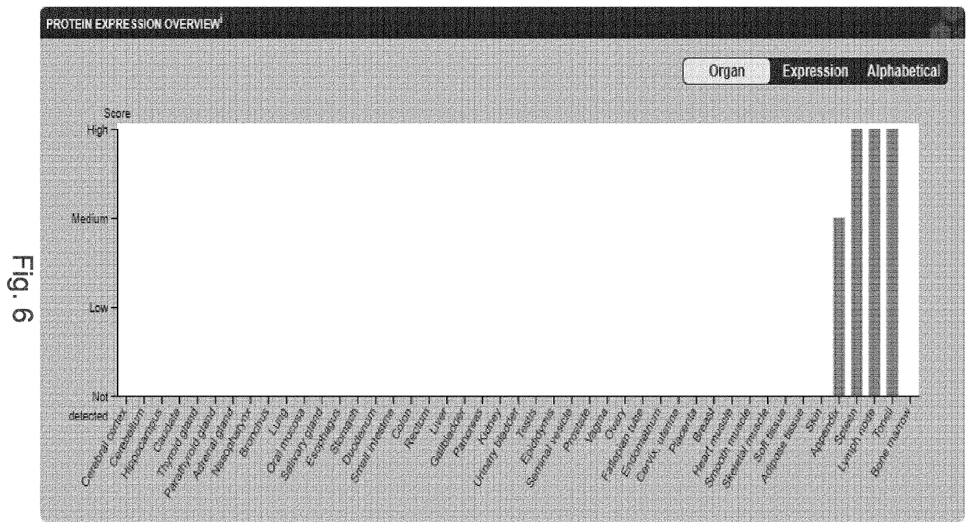

Analytical SEC reveals a low propensity to aggregation from humanized anti CD79B toxin fusion, between 3% (ATB-704) to 8% (ATB-508) of aggregates (FIG. 4). We observed than humanized anti CD79B toxin fusions exert a much lower aggregation propensity compared to the benchmark Polatuzumab (FIG. 5) which achieve 13% of aggregates (ATB-452)

### REFERENCES

Köhler, G. & Milstein, C. (1975): Continuous cultures of fused cells secreting antibody of predefined specificity. In: Nature. Bd. 256, S. 495-497. Jonsson and Malmquist, Advances in Biosnsors, 2:291-336 (1992)
Pluckthun and Skerra, Meth. Enzymol., 178:497-515 (1989) and in Day, E. D., Advanced Immunochemistry, Second Ed., Wiley-Liss, Inc., New York, N.Y. (1990)
Harding, The immunogenicity of humanized and fully human antibodies. MAbs. 2010 May-June; 2 (3): 256-265.
Eylenstein, et al, Molecular basis of in vitro affinity maturation and functional evolution of a neutralizing anti-human GM-CSF antibody, mAbs, 8:1, 176-186 (2016)
Kabat et al., J. Biol. Chem. 252, 6609-6616 (1977) and Kabat et al., Sequences of protein of immunological interest. (1991)
Chothia et al., J. Mol. Biol. 196:901-917 (1987)
Brühl H, Cihak J, Talke Y, Rodriguez Gomez M, Hermann F, Goebel N, Renner K, Plachý J, Stangassinger M, Aschermann S, Nimmerjahn F, Mack M. B-cell inhibition by cross-linking CD79b is superior to B-cell depletion with anti-CD20 antibodies in treating murine collagen-induced arthritis. Eur J Immunol. 2015 March; 45 (3): 705-15.
Tilly H et al. Polatuzumab Vedotin in Previously Untreated Diffuse Large B-Cell Lymphoma. N Engl J Med 2022; 386:351-363
Okazaki et al., Blood, 81:84-94 (1993)
Panowski S, Bhakta S, Raab H, Polakis P, Junutula JR. Site-specific antibody drug conjugates for cancer therapy. MAbs. 2014 January-Feb; 6 (1): 34-45. doi: 10.4161/mabs.27022. PMID: 24423619; PMCID: PMC3929453.
Steiner M, Neri D. Antibody-radionuclide conjugates for cancer therapy: historical considerations and new trends. Clin Cancer Res. 2011 Oct. 15; 17 (20): 6406-16

### Sequences

The following sequences form part of the disclosure of the present application. A WIPO ST 26 compatible electronic sequence listing is provided with this application, too. For the avoidance of doubt, if discrepancies exist between the sequences in the following table and the electronic sequence listing, the sequences in this table shall be deemed to be the correct ones. In some cases, the signal peptides may be encompassed in the reproduced sequences. In such case, the sequences shall be deemed disclosed with and without signal peptides. A readily available tool to identify signal peptides in a given protein sequence is SignalP-6.0 provided by Dansk Technical University.

Also note that in some embodiments, the respective amino acid sequence of the toxin shows a deimmunized version thereof. All embodiments shall be deemed to be disclosed with either the wildtype toxin sequence or the deimmunized variant.

TABLE 4
Sequences

| SEQ | | | |
|---|---|---|---|
| ID | | | |
| NO | Antibody | Domain | Sequence |
| 1 | 580 | VH | GAGGTTCAACTCGTTCAGTCTGGTGCAGAACTCAAGAAGC |
| | | | CTGGTGCTAGTGTTAAGGTGTCATGTAAAGCTAGTGGTTA |
| | | | TACTTTCTCTTCATATTGGATTGAATGGGTTAGACAAGCT |
| | | | CCTGGACAGGGTCTTGAGTGGATTGGAGAGATTTTGCCTG |
| | | | GTGGTGGAGATACAAACTACAATGAGATTTTTAAGGGTAG |
| | | | AGCTACTTTTACAGCTGATACTTCTACAAATACTGCTTAC |
| | | | ATGCAACTTTCTTCATTGAGATCAGAAGATACAGCTGTTT |
| | | | ATTACTGTACTAGAAGGGTTCCTGTTTTCTTTGATTACTG |
| | | | GGGACAGGGTACACTTGTTACTGTGTCATCT |
| 2 | 580 | VL | GCTATTCAATTAACTCAGTCTCCTTCAAGTCTTAGTGCTT |
| | | | CAGTTGGAGATAGGGTTACCATTACATGCAAGGCTAGTCA |
| | | | GTCTGTGGATTACGAAGGAGATTCTTTTCTTAACTGGTAC |
| | | | CAACAGAAGCCTGGTAAAGCTCCAAAGCTTTTGATCTATG |
| | | | CTGCTTCAAATTTGGAATCTGGAGTTCCTTCAAGATTTTC |
| | | | TGGTTCAGGATCTGGTACTGATTTCACTCTTACAATCTCT |
| | | | TCATTGCAACCAGAGGATTTTGCTACATACTTTTGTCAAC |
| | | | AGTCAAACGAGGATCCATTAACATTTGGTGGTGGTACTAA |
| | | | GTTGGAGATTAAA |
| 3 | 580 | VH | EVQLVQSGAELKKPGASVKVSCKASGYTFSSYWIEWVRQA |
| | | | PGQGLEWIGEILPGGGDTNYNEIFKGRATFTADTSTNTAY |
| | | | MQLSSLRSEDTAVYYCTRRVPVFFDYWGQGTLVTVSS |
| 4 | 580 | VL | AIQLTQSPSSLSASVGDRVTITCKASQSVDYEGDSFLNWY |
| | | | QQKPGKAPKLLIYAASNLESGVPSRFSGSGSGTDFTLTIS |
| | | | SLQPEDFATYFCQQSNEDPLTFGGGTKLEIK |
| 5 | 580 | HCDR1 | GYTFSSYWIE |
| 6 | 580 | HCDR2 | GEILPGGGDTNYNEIFKG |
| 7 | 580 | HCDR3 | TRRVPVFFDY |
| 8 | 580 | LCDR1 | KASQSVDYEGDSFLN |
| 9 | 580 | LCDR2 | AASNLES |
| 10 | 580 | LCDR3 | QQSNEDPLT |
| 11 | 693 | VH | GAGGTTCAGTTAGTTCAATCAGGTGCAGAATTAAAGAAAC |
| | | | CAGGTGCTTCAGTTAAAGTTAGTTGTAAGGCTAGTGGATA |
| | | | TACATTTTCTTCATATTGGATTGAATGGGTTAGACAAGCT |
| | | | CCTGGACAGGGTCTTGAGTGGATTGGAGAGATTTTGCCTG |
| | | | GTGGTGGAGATACAAACTACAATGAGATTTTTAAGGGTAG |
| | | | AGCTACTTTTACAGCTGATACTTCTACAAATACTGCTTAC |
| | | | ATGCAACTTTCTTCATTGAGATCAGAAGATACAGCTGTTT |
| | | | ATTACTGTACTAGAAGGGTTCCTATCTATCTTGATTACTG |
| | | | GGGACAGGGTACACTTGTTACTGTGTCTAGT |
| 12 | 693 | VL | GCTATTCAATTAACTCAGTCTCCTTCAAGTCTTAGTGCTT |
| | | | CAGTTGGAGATAGGGTTACCATTACATGCAAGGCTAGTCA |
| | | | GTCTGTGGATTACGAAGGAGATTCTTTTCTTAACTGGTAC |
| | | | CAACAGAAGCCTGGTAAAGCTCCAAAGCTTTTGATCTATG |
| | | | CTGCTTCAAATTTGGAATCTGGAGTTCCTTCAAGATTTTC |
| | | | TGGTTCAGGATCTGGTACTGATTTCACTCTTACAATCTCT |
| | | | TCATTGCAACCAGAGGATTTTGCTACATACTTTTGTCAAC |
| | | | AGTCAAACGAGGATCCATTAACATTTGGTGGTGGTACTAA |
| | | | GTTGGAGATTAAA |
| 13 | 693 | VH | EVQLVQSGAELKKPGASVKVSCKASGYTFSSYWIEWVRQA |
| | | | PGQGLEWIGEILPGGGDTNYNEIFKGRATFTADTSTNTAY |
| | | | MQLSSLRSEDTAVYYCTRRVPIYLDYWGQGTLVTVSS |
| 14 | 693 | VL | AIQLTQSPSSLSASVGDRVTITCKASQSVDYEGDSFLNWY |
| | | | QQKPGKAPKLLIYAASNLESGVPSRFSGSGSGTDFTLTIS |
| | | | SLQPEDFATYFCQQSNEDPLTFGGGTKLEIK |
| 15 | 693 | HCDR1 | GYTFSSYWIE |
| 16 | 693 | HCDR2 | GEILPGGGDTNYNEIFKG |
| 17 | 693 | HCDR3 | TRRVPIYLDY |
| 18 | 693 | LCDR1 | KASQSVDYEGDSFLN |
| 19 | 693 | LCDR2 | AASNLES |
| 20 | 693 | LCDR3 | QQSNEDPLT |
| 21 | 697 | VH | GAGGTTCAGTTAGTTCAATCAGGTGCAGAATTAAAGAAAC |
| | | | CAGGTGCTTCAGTTAAAGTTAGTTGTAAGGCTAGTGGATA |
| | | | TACATTTTCTTCATATTGGATTGAATGGGTTAGACAAGCT |
| | | | CCTGGACAGGGTCTTGAGTGGATTGGAGAGATTTTGCCTG |
| | | | GTGGTGGAGATACAAACTACAATGAGATTTTTAAGGGTAG |
| | | | AGCTACTTTTACAGCTGATACTTCTACAAATACTGCTTAC |
| | | | ATGCAACTTTCTTCATTGAGATCAGAAGATACAGCTGTTT |
| | | | ATTACTGTACTAGAAGGGTTCCTATTTTTCTTGATTACTG |
| | | | GGGACAGGGTACACTTGTTACTGTGTCTAGT |
| 22 | 697 | VL | GCTATTCAATTAACTCAGTCTCCTTCAAGTCTTAGTGCTT |
| | | | CAGTTGGAGATAGGGTTACCATTACATGCAAGGCTAGTCA |
| | | | GTCTGTGGATTACGAAGGAGATTCTTTTCTTAACTGGTAC |
| | | | CAACAGAAGCCTGGTAAAGCTCCAAAGCTTTTGATCTATG |
| | | | CTGCTTCAAATTTGGAATCTGGAGTTCCTTCAAGATTTTC |
| | | | TGGTTCAGGATCTGGTACTGATTTCACTCTTACAATCTCT |
| | | | TCATTGCAACCAGAGGATTTTGCTACATACTTTTGTCAAC |
| | | | AGTCAAACGAGGATCCATTAACATTTGGTGGTGGTACTAA |
| | | | GTTGGAGATTAAA |
| 23 | 697 | VH | EVQLVQSGAELKKPGASVKVSCKASGYTFSSYWIEWVRQA |
| | | | PGQGLEWIGEILPGGGDTNYNEIFKGRATFTADTSTNTAY |
| | | | MQLSSLRSEDTAVYYCTRRVPIFLDYWGQGTLVTVSS |
| 24 | 697 | VL | AIQLTQSPSSLSASVGDRVTITCKASQSVDYEGDSFLNWY |
| | | | QQKPGKAPKLLIYAASNLESGVPSRFSGSGSGTDFTLTIS |
| | | | SLQPEDFATYFCQQSNEDPLTFGGGTKLEIK |
| 25 | 697 | HCDR1 | GYTFSSYWIE |
| 26 | 697 | HCDR2 | GEILPGGGDTNYNEIFKG |
| 27 | 697 | HCDR3 | TRRVPIFLDY |
| 28 | 697 | LCDR1 | KASQSVDYEGDSFLN |
| 29 | 697 | LCDR2 | AASNLES |
| 30 | 697 | LCDR3 | QQSNEDPLT |
| 31 | 704 | VH | GAGGTTCAGTTAGTTCAATCAGGTGCAGAATTAAAGAAAC |
| | | | CAGGTGCTTCAGTTAAAGTTAGTTGTAAGGCTAGTGGATA |
| | | | TACATTTTCTTCATATTGGATTGAATGGGTTAGACAAGCT |
| | | | CCTGGACAGGGTCTTGAGTGGATTGGAGAGATTTTGCCTG |
| | | | GTGGTGGAGATACAAACTACAATGAGATTTTTAAGGGTAG |
| | | | AGCTACTTTTACAGCTGATACTTCTACAAATACTGCTTAC |
| | | | ATGCAACTTTCTTCATTGAGATCAGAAGATACAGCTGTTT |
| | | | ATTACTGTACTAGAAGGGTTCCTATTAAGTTGGATTACTG |
| | | | GGGACAGGGTACACTTGTTACTGTGTCTAGT |
| 32 | 704 | VL | GCTATTCAATTAACTCAGTCTCCTTCAAGTCTTAGTGCTT |
| | | | CAGTTGGAGATAGGGTTACCATTACATGCAAGGCTAGTCA |
| | | | GTCTGTGGATTACGAAGGAGATTCTTTTCTTAACTGGTAC |
| | | | CAACAGAAGCCTGGTAAAGCTCCAAAGCTTTTGATCTATG |
| | | | CTGCTTCAAATTTGGAATCTGGAGTTCCTTCAAGATTTTC |
| | | | TGGTTCAGGATCTGGTACTGATTTCACTCTTACAATCTCT |
| | | | TCATTGCAACCAGAGGATTTTGCTACATACTTTTGTCAAC |
| | | | AGTCAAACGAGGATCCATTAACATTTGGTGGTGGTACTAA |
| | | | GTTGGAGATTAAA |
| 33 | 704 | VH | EVQLVQSGAELKKPGASVKVSCKASGYTFSSYWIEWVRQA |
| | | | PGQGLEWIGEILPGGGDTNYNEIFKGRATFTADTSTNTAY |
| | | | MQLSSLRSEDTAVYYCTRRVPIKLDYWGQGTLVTVSS |
| 34 | 704 | VL | AIQLTQSPSSLSASVGDRVTITCKASQSVDYEGDSFLNWY |
| | | | QQKPGKAPKLLIYAASNLESGVPSRFSGSGSGTDFTLTIS |
| | | | SLQPEDFATYFCQQSNEDPLTFGGGTKLEIK |
| 35 | 704 | HCDR1 | GYTFSSYWIE |
| 36 | 704 | HCDR2 | GEILPGGGDTNYNEIFKG |
| 37 | 704 | HCDR3 | TRRVPIKLDY |
| 38 | 704 | LCDR1 | KASQSVDYEGDSFLN |
| 39 | 704 | LCDR2 | AASNLES |
| 40 | 704 | LCDR3 | QQSNEDPLT |
| 41 | CD79b isoform 1 | NP_000617.1 | MARLALSPVPSHWMVALLLLLSAEPVPAARSEDRYRNPKG |
| | | | SACSRIWQSPRFIARKRGFTVKMHCYMNSASGNVSWLWKQ |
| | | | EMDENPQQLKLEKGRMEESQNESLATLTIQGIRFEDNGIY |
| | | | FCQQKCNNTSEVYQGCGTELRVMGFSTLAQLKQRNTLKDG |
| | | | IIMIQTLLIILFIIVPIFLLLDKDDSKAGMEEDHTYEGLD |
| | | | IDQTATYEDIVTLRTGEVKWSVGEHPGQE |
| 42 | CD79b isoform 3 | NP_001035022.1 | MARLALSPVPSHWMVALLLLLSAAEPVPAARSEDRYRNPK |
| | | | GSACSRIWQSPRFIARKRGFTVKMHCYMNSASGNVSWLWK |
| | | | QEMDENPQQLKLEKGRMEESQNESLATLTIQGIRFEDNGI |
| | | | YFCQQKCNNTSEVYQGCGTELRVMGFSTLAQLKQRNTLKD |
| | | | GIIMIQTLLIILFIIVPIFLLLDKDDSKAGMEEDHTYEGL |
| | | | DIDQTATYEDIVTLRTGEVKWSVGEHPGQE |
| 43 | CD79b isoform 4 | NP_001315979.1 | MARLALSPVPSHWMVALLLLLSAAEPVPAARSEDRYRNPK |
| | | | GFSTLAQLKQRNTLKDGIIMIQTLLIILFIIVPIFLLLDK |
| | | | DDSKAGMEEDHTYEGLDIDQTATYEDIVTLRTGEVKWSVG |
| | | | EHPGQE |
| 44 | CD79b isoform 2 | NP_067613.1 | MARLALSPVPSHWMVALLLLLSAEPVPAARSEDRYRNPKGFSTLA |
| | | | QLKQRNTLKDGIIMIQTLLIILFIIVPIFLLLDKDDSKAGMEEDH |
| | | | TYEGLDIDQTATYEDIVTLRTGEVKWSVGEHPGQE |
| 45 | Fpe liker | | TRHRQPRGWEQL |
| 46 | Fdt linker | | AGNRVRRSVG |
| 47 | Furin Cleavage | | HRRRKRSLDTS |
| | site (FCS)/Liop | | |
| 48 | hirsutelin A | | APIVTCRPKLDGREKPFKVDVATAQAQARKAGLTTGKSGDPHRYF |
| | | | AGDHIRWGVNNCDKADAILWEYPIYWVGKNAEWAKDVKTSQQKGG |
| | | | PTPIRVVYANSRGAVQYCGVMTHSKVDKNNQGKEFFEKCD |
| 49 | Anisoplin | | ADTVICEPHQDTETGKIKKFKVDVGVAEDQAKKAGLTTGKSGDPH |
| | | | RYMNGDKINFGIHNCDKEGAILWEYPIYWVGKKAEWMKDEKTDRQ |
| | | | PGGPTPIRVVYANNNGNIVYCGVMTHAVVKSNNQGEKFFLKCT |
| 50 | Anisoplin | | ADTVICEPHQDTETGKIKKFKVDVGVAEDQAKKAGLTTGKSGYPH |
| | (deimmunized | | RYMNGDKINFGIHNCDKEGAILWEYPIYWVGKKAEWMKDEKTDRQ |
| | version D43Y) | | PGGPTPIRVVYANNNGNIVYCGVMTHAVVKSNNQGEKFFLKCT |
| 51 | Angiogenin | | QDNSRYTHFLTQHYDAKPQGRDDRYCESIMRRRGLTSPCKDINTF |
| | | | IHGNKRSIKAICENKNGNPHRENLRISKSSFQVTTCKLHRRSPWP |
| | | | PCQYRATAGFRNVVVACENGLPVHLDQSIFRRP |
| 52 | Anisoplin | | ADTVICEPHQDTETGKIKKFKVDVGVAEDQAKKAGLITGKSGDPH |
| | homologue 1 | | RYMNGDKINFGIHNCDKEGAILWEYPIYWVGKKAEWMKDEKTDRQ |
| | | | PGGPTPIRVVYANNNGNIVYCGVMTHAVVKSNNQGEKFFLKCT |
| 53 | Anisoplin | | ADTVICEPRQDTETGKIKKFKVDVGVAEDQAKKAGLITGKSGDPH |
| | homologue 2 | | RYMNGDKINFGIHNCDKEGAILWEYPIYWVGKKAEWMKDEKTDRQ |
| | | | PGGPTPIRVVYANNNGNIVYCGVMTHAVVKSNNQGEKFFLKCT |
| 54 | Anisoplin | | ADTVICEPHQDTETGKIKKFTVDVGVAEDQAKKAGLTTGKSGDPH |
| | homologue 3 | | RYMNGDNINFGIHNCDKEGAILWEYPIYWVGKKAEWKKDEKTDRQ |
| | | | PGGPTPLRVVYANNNGNIVYCGVMTHAVVKSNNQGEKFFLKCT |
| 55 | Ageritin | | AETVQYYNSYSDASIASCAFVDSGKDKIDKTKLVTYTSRLAASPA |
| | | | YQKVVGVGLKTAAGSIVPYVRLDMDNTGKGIHFNATKLSDSSAKL |
| | | | AAVLKTTVSMTEAQRTQLYMEYIKGIENRSAQFIWDWWRTGKAPA |
| 56 | α-Sarcin | | AVTWTCLNDQKNPKTNKYETKRLLYNQNKAESNSHHAPLSDGKTG |
| | | | SSYPHWFTNGYDGDGKLPKGRTPIKFGKSDCDRPPKHSKDGNGKT |
| | | | DHYLLEFPTFPDGHDYKFDSKKPKENPGPARVIYTYPNKVFCGII |
| | | | AHTKENQGELKLCSH |
| 57 | α-Sarcin | | AVTWTCLNTQKNPKTNKYETKRLLYNQNKAESNSHHAPLSDGKTG |
| | (deimmunized | | SSYPHWFTNGYDGDGKLPKGRTPIKFGKSDCDRPPKHSKDGNGKT |
| | version | | DHYLLEFPTFPDGHDYKFDSKKPKENPGPARVIYTYPNKVFCGII |
| | D9T/Q142T) | | AHTKENTGELKLCSH |
| 58 | hpRNase1 | | KESRAKKFQRQHMDSDSSPSSSSTYCNQMMRRRNMTQGRCKPVNT |
| | | | FVHEPLVDVQNVCFQEKVTCKNGQGNCYKSNSSMHITDCRLTNGS |
| | | | RYPNCAYRTSPKERHIIVACEGSPYVPVHFDASVEDST |
| 59 | restrictocin | | MVAIKNLFLLAATAVSVLAAPSPLDARATWTCINQQLNPKTNKWE |
| | | | DKRLLYSQAKAESNSHHAPLSDGKTGSSYPHWFTNGYDGNGKLIK |
| | | | GRTPIKFGKADCDRPPKHSQNGMGKDDHYLLEFPTFPDGHDYKFD |
| | | | SKKPKEDPGPARVIYTYPNKVFCGIVAHQRGNQGDLRLCSH |
| 60 | G4S linker | | GGGGS |
| 61 | epitope of CD79b | | ARSEDRYRNPKGSACSRIWQS |

What is claimed is:

1. An antibody or a target-binding fragment or derivative thereof retaining target binding capacity to human CD79b, which
a) comprises a set of six heavy chain/light chain complementarity determining regions (CDR) comprised in the heavy chain/light variable domain sequence pair selected from one of the following pairs of SEQ ID NOs:
3 and 4,
13 and 14,
23 and 24, or
33 and 34,
b) comprises a set of six heavy chain/light chain complementarity determining regions (CDR) selected from, in the order HCDR1; HCDR2; HCDR3; LCDR1; LCDR2 and LCDR3,
i) SEQ ID NOs: 5, 6, 7, 8, 9 and 10
ii) SEQ ID NOs: 15, 16, 17, 18, 19 and 20
iii) SEQ ID NOs: 25, 26, 27, 28, 29 and 30, or
iv) SEQ ID NOs: 35, 36, 37, 38, 39 and 40,
c) comprises a set of heavy chain/light chain complementarity determining regions (CDR) as set forth in option b), with the proviso that at least one of the CDRs has up to 3 amino acid substitutions relative to the CDRs comprised in the respective SEQ ID NOs, and/or
d) comprises a set of heavy chain/light chain complementarity determining regions (CDR) as set forth in option b) or c), with the proviso that at least one of the CDRs has a sequence identity of ≥66% to the CDRs comprised in the respective SEQ ID NOS,
wherein the CDRs are embedded in a suitable protein framework so as to be capable to bind to human CD79b.

2. The antibody or fragment of claim 1, wherein the antibody is a humanized antibody or fragment.

3. The antibody or fragment according to claim 1, which comprises
a) the heavy chain/light chain variable domain (HCVD/LCVD) pairs set forth in the following pairs of SEQ ID NOs:
3 and 4,
13 and 14,
23 and 24, or
33 and 34,
b) the heavy chain/light chain variable domains (HCVD/LCVD) pairs of a), with the proviso that
the HCVD has a sequence identity of ≥80% to the HCVD comprised in the respective SEQ ID NO, and/or
the LCVD has a sequence identity of ≥80% to the LCVD comprised in the respective SEQ ID NO,
c) the heavy chain/light chain variable domains (HCVD/LCVD) pairs as set forth in option a) or b), with the proviso that at least one of the HCVD or LCVD has up to 10 amino acid substitutions relative to the HCVD or LCVD comprised in the respective SEQ ID NO,
said antibody or fragment being capable to bind to human CD79b.

4. The antibody or fragment according to claim 1, wherein at least one amino acid substitution is a conservative amino acid substitution.

5. The antibody or fragment according to claim 1, wherein the human CD79b to which the antibody or fragment binds, comprises
a) the amino acid sequence set forth in SEQ ID NO: 41-44 or
b) an amino acid sequence that has at least 80% sequence identity with SEQ ID NO: 41-44.

6. The antibody or fragment according to claim 1, which is a monoclonal antibody, or a target-binding fragment or derivative thereof retaining target binding capacity to human CD79b.

7. The antibody or fragment according to claim 1, which is in at least one of the formats selected from the group consisting of: IgG, scFv, Fab, or (Fab) 2.

8. A nucleic acid that encodes for at least one chain of antibody or fragment according to claim 1.

9. A recombinant immunotoxin, an immunocytokine, an antibody drug conjugate or an antibody-radionucleide conjugate comprising the antibody or fragment according to claim 1.

10. A pharmaceutical composition comprising the antibody or fragment according to claim 1 and one or more pharmaceutically acceptable excipients.

11. A combination comprising (i) the antibody or fragment according to claim 1 and (ii) one or more therapeutically active compounds.

12. (canceled)

13. A method for treating or preventing a neoplastic disease, which method comprises administration, to a human or animal subject, of the antibody or fragment according to claim 1 in a therapeutically sufficient dose.

14. A therapeutic kit of parts comprising:
a) the antibody or fragment according to claim 1,
b) an apparatus for administering the composition, composition or combination, and
c) instructions for use.

15. A method for treating or preventing a neoplastic disease, which method comprises administration, to a human or animal subject, the recombinant immunotoxin, immunocytokine, antibody drug conjugate or antibody-radionucleide according to claim 9 in a therapeutically sufficient dose.


SEQUENCE LISTING (61 sequences)

SEQ ID NO: 1 - DNA, 351 bp, synthetic construct
  source 1..351: mol_type = other DNA
gaggttcaactcgttcagtctggtgcagaactcaagaagcctggtgctagtgttaaggtgtcatgtaaagctagtggttatactttctcttcatattggattgaatgggttagacaagctcctggacagggtcttgagtggattggagagattttgcctggtggtggagatacaaactacaatgagatttttaagggtagagctacttttacagctgatacttctacaaatactgcttacatgcaactttcttcattgagatcagaagatacagctgtttattactgtactagaagggttcctgttttctttgattactggggacagggtacacttgttactgtgtcatct

SEQ ID NO: 2 - DNA, 333 bp, synthetic construct
  source 1..333: mol_type = other DNA
gctattcaattaactcagtctccttcaagtcttagtgcttcagttggagatagggttaccattacatgcaaggctagtcagtctgtggattacgaaggagattcttttcttaactggtaccaacagaagcctggtaaagctccaaagcttttgatctatgctgcttcaaatttggaatctggagttccttcaagattttctggttcaggatctggtactgatttcactcttacaatctcttcattgcaaccagaggattttgctacatacttttgtcaacagtcaaacgaggatccattaacatttggtggtggtactaagttggagattaaa

SEQ ID NO: 3 - protein, 117 aa, synthetic construct
EVQLVQSGAELKKPGASVKVSCKASGYTFSSYWIEWVRQAPGQGLEWIGEILPGGGDTNYNEIFKGRATFTADTSTNTAYMQLSSLRSEDTAVYYCTRRVPVFFDYWGQGTLVTVSS

SEQ ID NO: 4 - protein, 111 aa, synthetic construct
AIQLTQSPSSLSASVGDRVTITCKASQSVDYEGDSFLNWYQQKPGKAPKLLIYAASNLESGVPSRFSGSGSGTDFTLTISSLQPEDFATYFCQQSNEDPLTFGGGTKLEIK

SEQ ID NO: 5 - protein, 10 aa, synthetic construct
GYTFSSYWIE

SEQ ID NO: 6 - protein, 18 aa, synthetic construct
GEILPGGGDTNYNEIFKG

SEQ ID NO: 7 - protein, 10 aa, synthetic construct
TRRVPVFFDY

SEQ ID NO: 8 - protein, 15 aa, synthetic construct
KASQSVDYEGDSFLN

SEQ ID NO: 9 - protein, 7 aa, synthetic construct
AASNLES

SEQ ID NO: 10 - protein, 9 aa, synthetic construct
QQSNEDPLT

SEQ ID NO: 11 - DNA, 351 bp, synthetic construct
  source 1..351: mol_type = other DNA
gaggttcagttagttcaatcaggtgcagaattaaagaaaccaggtgcttcagttaaagttagttgtaaggctagtggatatacattttcttcatattggattgaatgggttagacaagctcctggacagggtcttgagtggattggagagattttgcctggtggtggagatacaaactacaatgagatttttaagggtagagctacttttacagctgatacttctacaaatactgcttacatgcaactttcttcattgagatcagaagatacagctgtttattactgtactagaagggttcctatctatcttgattactggggacagggtacacttgttactgtgtctagt

SEQ ID NO: 12 - DNA, 333 bp, synthetic construct; identical to SEQ ID NO: 2
  source 1..333: mol_type = other DNA

SEQ ID NO: 13 - protein, 117 aa, synthetic construct
EVQLVQSGAELKKPGASVKVSCKASGYTFSSYWIEWVRQAPGQGLEWIGEILPGGGDTNYNEIFKGRATFTADTSTNTAYMQLSSLRSEDTAVYYCTRRVPIYLDYWGQGTLVTVSS

SEQ ID NO: 14 - protein, 111 aa, synthetic construct; identical to SEQ ID NO: 4

SEQ ID NO: 15 - protein, 10 aa, synthetic construct
GYTFSSYWIE

SEQ ID NO: 16 - protein, 18 aa, synthetic construct
GEILPGGGDTNYNEIFKG

SEQ ID NO: 17 - protein, 10 aa, synthetic construct
TRRVPIYLDY

SEQ ID NO: 18 - protein, 15 aa, synthetic construct
KASQSVDYEGDSFLN

SEQ ID NO: 19 - protein, 7 aa, synthetic construct
AASNLES

SEQ ID NO: 20 - protein, 9 aa, synthetic construct
QQSNEDPLT

SEQ ID NO: 21 - DNA, 351 bp, synthetic construct
  source 1..351: mol_type = other DNA
gaggttcagttagttcaatcaggtgcagaattaaagaaaccaggtgcttcagttaaagttagttgtaaggctagtggatatacattttcttcatattggattgaatgggttagacaagctcctggacagggtcttgagtggattggagagattttgcctggtggtggagatacaaactacaatgagatttttaagggtagagctacttttacagctgatacttctacaaatactgcttacatgcaactttcttcattgagatcagaagatacagctgtttattactgtactagaagggttcctatttttcttgattactggggacagggtacacttgttactgtgtctagt

SEQ ID NO: 22 - DNA, 333 bp, synthetic construct; identical to SEQ ID NO: 2
  source 1..333: mol_type = other DNA

SEQ ID NO: 23 - protein, 117 aa, synthetic construct
EVQLVQSGAELKKPGASVKVSCKASGYTFSSYWIEWVRQAPGQGLEWIGEILPGGGDTNYNEIFKGRATFTADTSTNTAYMQLSSLRSEDTAVYYCTRRVPIFLDYWGQGTLVTVSS

SEQ ID NO: 24 - protein, 111 aa, synthetic construct; identical to SEQ ID NO: 4

SEQ ID NO: 25 - protein, 10 aa, synthetic construct
GYTFSSYWIE

SEQ ID NO: 26 - protein, 18 aa, synthetic construct
GEILPGGGDTNYNEIFKG

SEQ ID NO: 27 - protein, 10 aa, synthetic construct
TRRVPIFLDY

SEQ ID NO: 28 - protein, 15 aa, synthetic construct
KASQSVDYEGDSFLN

SEQ ID NO: 29 - protein, 7 aa, synthetic construct
AASNLES

SEQ ID NO: 30 - protein, 9 aa, synthetic construct
QQSNEDPLT

SEQ ID NO: 31 - DNA, 351 bp, synthetic construct
  source 1..351: mol_type = other DNA
gaggttcagttagttcaatcaggtgcagaattaaagaaaccaggtgcttcagttaaagttagttgtaaggctagtggatatacattttcttcatattggattgaatgggttagacaagctcctggacagggtcttgagtggattggagagattttgcctggtggtggagatacaaactacaatgagatttttaagggtagagctacttttacagctgatacttctacaaatactgcttacatgcaactttcttcattgagatcagaagatacagctgtttattactgtactagaagggttcctattaagttggattactggggacagggtacacttgttactgtgtctagt

SEQ ID NO: 32 - DNA, 333 bp, synthetic construct; identical to SEQ ID NO: 2
  source 1..333: mol_type = other DNA

SEQ ID NO: 33 - protein, 117 aa, synthetic construct
EVQLVQSGAELKKPGASVKVSCKASGYTFSSYWIEWVRQAPGQGLEWIGEILPGGGDTNYNEIFKGRATFTADTSTNTAYMQLSSLRSEDTAVYYCTRRVPIKLDYWGQGTLVTVSS

SEQ ID NO: 34 - protein, 111 aa, synthetic construct; identical to SEQ ID NO: 4

SEQ ID NO: 35 - protein, 10 aa, synthetic construct
GYTFSSYWIE

SEQ ID NO: 36 - protein, 18 aa, synthetic construct
GEILPGGGDTNYNEIFKG

SEQ ID NO: 37 - protein, 10 aa, synthetic construct
TRRVPIKLDY

SEQ ID NO: 38 - protein, 15 aa, synthetic construct
KASQSVDYEGDSFLN

SEQ ID NO: 39 - protein, 7 aa, synthetic construct
AASNLES

SEQ ID NO: 40 - protein, 9 aa, synthetic construct
QQSNEDPLT

SEQ ID NO: 41 - protein, 229 aa, Homo sapiens
MARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 42 - protein, 230 aa, Homo sapiens
MARLALSPVPSHWMVALLLLLSAAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 43 - protein, 126 aa, Homo sapiens
MARLALSPVPSHWMVALLLLLSAAEPVPAARSEDRYRNPKGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 44 - protein, 125 aa, Homo sapiens
MARLALSPVPSHWMVALLLLLSAEPVPAARSEDRYRNPKGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 45 - protein, 12 aa, synthetic construct
TRHRQPRGWEQL

SEQ ID NO: 46 - protein, 10 aa, synthetic construct
AGNRVRRSVG

SEQ ID NO: 47 - protein, 11 aa, synthetic construct
HRRRKRSLDTS

SEQ ID NO: 48 - protein, 130 aa, Hirsutella thompsonii
APIVTCRPKLDGREKPFKVDVATAQAQARKAGLTTGKSGDPHRYFAGDHIRWGVNNCDKADAILWEYPIYWVGKNAEWAKDVKTSQQKGGPTPIRVVYANSRGAVQYCGVMTHSKVDKNNQGKEFFEKCD

SEQ ID NO: 49 - protein, 133 aa, Metarhizium anisopliae
ADTVICEPHQDTETGKIKKFKVDVGVAEDQAKKAGLTTGKSGDPHRYMNGDKINFGIHNCDKEGAILWEYPIYWVGKKAEWMKDEKTDRQPGGPTPIRVVYANNNGNIVYCGVMTHAVVKSNNQGEKFFLKCT

SEQ ID NO: 50 - protein, 133 aa, synthetic construct
ADTVICEPHQDTETGKIKKFKVDVGVAEDQAKKAGLTTGKSGYPHRYMNGDKINFGIHNCDKEGAILWEYPIYWVGKKAEWMKDEKTDRQPGGPTPIRVVYANNNGNIVYCGVMTHAVVKSNNQGEKFFLKCT

SEQ ID NO: 51 - protein, 123 aa, Homo sapiens
QDNSRYTHFLTQHYDAKPQGRDDRYCESIMRRRGLTSPCKDINTFIHGNKRSIKAICENKNGNPHRENLRISKSSFQVTTCKLHRRSPWPPCQYRATAGFRNVVVACENGLPVHLDQSIFRRP

SEQ ID NO: 52 - protein, 133 aa, synthetic construct
ADTVICEPHQDTETGKIKKFKVDVGVAEDQAKKAGLITGKSGDPHRYMNGDKINFGIHNCDKEGAILWEYPIYWVGKKAEWMKDEKTDRQPGGPTPIRVVYANNNGNIVYCGVMTHAVVKSNNQGEKFFLKCT

SEQ ID NO: 53 - protein, 133 aa, synthetic construct
ADTVICEPRQDTETGKIKKFKVDVGVAEDQAKKAGLITGKSGDPHRYMNGDKINFGIHNCDKEGAILWEYPIYWVGKKAEWMKDEKTDRQPGGPTPIRVVYANNNGNIVYCGVMTHAVVKSNNQGEKFFLKCT

SEQ ID NO: 54 - protein, 133 aa, synthetic construct
ADTVICEPHQDTETGKIKKFTVDVGVAEDQAKKAGLTTGKSGDPHRYMNGDNINFGIHNCDKEGAILWEYPIYWVGKKAEWKKDEKTDRQPGGPTPLRVVYANNNGNIVYCGVMTHAVVKSNNQGEKFFLKCT

SEQ ID NO: 55 - protein, 135 aa, Agrocybe aegerita
AETVQYYNSYSDASIASCAFVDSGKDKIDKTKLVTYTSRLAASPAYQKVVGVGLKTAAGSIVPYVRLDMDNTGKGIHFNATKLSDSSAKLAAVLKTTVSMTEAQRTQLYMEYIKGIENRSAQFIWDWWRTGKAPA

SEQ ID NO: 56 - protein, 150 aa, Aspergillus giganteus
AVTWTCLNDQKNPKTNKYETKRLLYNQNKAESNSHHAPLSDGKTGSSYPHWFTNGYDGDGKLPKGRTPIKFGKSDCDRPPKHSKDGNGKTDHYLLEFPTFPDGHDYKFDSKKPKENPGPARVIYTYPNKVFCGIIAHTKENQGELKLCSH

SEQ ID NO: 57 - protein, 150 aa, synthetic construct
AVTWTCLNTQKNPKTNKYETKRLLYNQNKAESNSHHAPLSDGKTGSSYPHWFTNGYDGDGKLPKGRTPIKFGKSDCDRPPKHSKDGNGKTDHYLLEFPTFPDGHDYKFDSKKPKENPGPARVIYTYPNKVFCGIIAHTKENTGELKLCSH

SEQ ID NO: 58 - protein, 128 aa, Homo sapiens
KESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 59 - protein, 176 aa, Aspergillus restrictus
MVAIKNLFLLAATAVSVLAAPSPLDARATWTCINQQLNPKTNKWEDKRLLYSQAKAESNSHHAPLSDGKTGSSYPHWFTNGYDGNGKLIKGRTPIKFGKADCDRPPKHSQNGMGKDDHYLLEFPTFPDGHDYKFDSKKPKEDPGPARVIYTYPNKVFCGIVAHQRGNQGDLRLCSH

SEQ ID NO: 60 - protein, 5 aa, synthetic construct
GGGGS

SEQ ID NO: 61 - protein, 21 aa, Homo sapiens
ARSEDRYRNPKGSACSRIWQS